SEQUENCE LISTING (36 sequences)

SEQ ID NO: 1 - DNA, 34 bp, Artificial Sequence
  primer
cggagctctatgttaattgcttccacaatgggac

SEQ ID NO: 2 - DNA, 21 bp, Artificial Sequence
  primer
gcattctacttctattgcagc

SEQ ID NO: 3 - DNA, 40 bp, Artificial Sequence
  primer
tagctagcagaaggcatgtggttgtgactccgaggggttg

SEQ ID NO: 4 - DNA, 212 bp, bean yellow dwarf virus
taggttgccagtctgatttcactgtcaaccctaaatatggaaaaaagaagaaaataaaaggtgggatcccttctataattctttggaatcctgacgaagactggatgttatcaatgacaagtcaacagaaggattactttgaagataattgcgtcacccactatatgtgtgacggggagactttttttgctcgggaatcgtcgagtcactga

SEQ ID NO: 5 - DNA, 1091 bp, bean yellow dwarf virus
atgccttctgctagcaagaacttcagactccaatctaaatatgttttccttacctaccccaagtgctcatctcaaagagatgatttattccagtttctctgggagaaactcacaccttttcttattttcttccttggtgttgcttctgagcttcatcaagatggcactacccactatcatgctcttatccagcttgataaaaaaccttgtattagggatccttcttttttcgattttgaaggaaatcaccctaatatccagccagctagaaactctaaacaagtccttgattacatatcaaaggacggagatattaaaaccagaggagatttccgagatcataaggtctctcctcgcaaatctgacgcacgatggcgaactattatccagactgcaacgtctaaggaggagtatcttgacatgatcaaagaagaattccctcatgaatgggcaacaaagcttcaatggctggaatattcagccaacaaattatttcctccacaacctgagcagtacgtgtcgcccttcacagaatcagatctccgctgccacgaagatctgcacaactggagagagacgcacctatatcatgtaagcatcgatgcctacactttcatacatcctgtctcctacgatcaagcacaatctgaccttgagtggatggccgatctaaccaggatgagggaaggactggggtcagacaccccagcctctacatctgcggaccaactcgtaccggaaagaccacctgggctagaagtctcgggcgacacaactactggaacgggaccatcgacttcaccaactacgatgaacacgccacctataatatcatcgacgacatccccttcaagttcgtcccattgtggaagcaattaataggttgccagtctgatttcactgtcaaccctaaatatggaaaaaagaagaaaataaaaggtgggatcccttctataattctttggaatcctgacgaagactggatgttatcaatgacaagtcaacagaaggattactttgaagataattgcgtcacccactatatgtgtgacggggagactttttttgctcgggaatcgtcgagtcactga

SEQ ID NO: 6 - DNA, 76 bp, Tobacco mosaic virus
tcgagtatttttacaacaattaccaacaacaacaaacaacaaacaacattacaattactatttacaatctagaaca

SEQ ID NO: 7 - DNA, 79 bp, Nicotiana benthamiana
ctcgagaaacaaacaaaatcaacaaatatagaaaataacgcatttccaattctttgaaatttctgcaacatctagaaca

SEQ ID NO: 8 - DNA, 517 bp, Cowpea mosaic virus
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaattcgcgaccggt

SEQ ID NO: 9 - DNA, 251 bp, Cowpea mosaic virus
tagctcgaggcctttaactctggtttcattaaattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcag

SEQ ID NO: 10 - DNA, 144 bp, Barley yellow dwarf virus
tcgagtgaagattgaccatctcacaaaagctgttacgtgcttgtaacacactacacactcgttttgtattcgagaagtagttgcaacaacggtccccttattgcctgacaagctgagggccacccttctatccccaccgccacc

SEQ ID NO: 11 - DNA, 881 bp, Barley yellow dwarf virus
ggtaccagtgaagacaacaccactagcacaaatcggatcctgggaaacaggcagaacttcggttcataagctcgggtaggctgtcaacctaccgccgtatcgtattgtgtttggccgatggaggatcttcacgttatcgccgtttgtattcttgccttgactgtgctctctggggtaggcgctgttttgagttgctgccgttggtgctgcagcaatccttttcctccctccctctcttctgtttaagcaaaagactctcgatctgtgcgagagacaatcaaaaatatcgagggagcttcggctcagtgaggggattaacgacccccagtaatggccggtcctggcggacataaataacccgctataggacgaagtggtagccaccactgatcaaatggcaaacatgcttctgtgttgtacactgccccggagcctaccgggtcaacaaggctatcccaccaacccgatgaaatgagggtggagtgagcggagtgggtgacttcgtgatgtacacccgatcgtcaggattgaagacgttaaaactcgacgacctggtacaagtcgttaaactgactcgggtggatacaccacacccggcccagcatgttggcatacccacgatacgaaacgtgggtctcttggagccactacctgtgatgcaaggtagggtatgagtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccctcggggtactcttaacgctgacactcgaaagagcagttcggcaacccgagctc

SEQ ID NO: 12 - DNA, 97 bp, Pea enation mosaic virus
ctcgagggtatttatagagatcagtatgaactgtgtcgctaggatcaagcggtggttcacacctgacttcacccctggcgagggcgtgaagtctacc

SEQ ID NO: 13 - DNA, 710 bp, Pea enation mosaic virus
ggcttcgcttcccgccggaagaccgcggcggttctgttcctcccacaggagtacggcaacaacccaccttgggaaagtggggaccccagcactaactcctttaactaggcgggcgtgttggttacagtaggaggggacagtgcgcatcgaaactgagccccaccacaactctcatccacggggtggttgggacgcaggtgtcggagggatcgccagccctcaggatagtgagctcccgcagagggataagctatctccctgcgacgtagtggtagaacacgtgggataggggatgaccttgtcgaccggttatcggtcccctgctccttcgagctggcaaggcgctcacaggttctacactgctactaaagttggtggtggatgtctcgcccaaaaagatcacaaacgcgcgggacaaggtcccttccaccttcgccgggtaaggctagagtcagcgctgcatgactataacttgcggccgatccagttgcacgactggtggtccccctcagtgtctcggttgtctgccgagtgggcggtggtcggattccaccacaccctgccacgaggtgcgtggagacttggccagtctaggctcgtcgtaattagttgcagcgacgttaatcaacccgtccgggcatataataggaccggttgtgcttcttcctcccttcttagccaggtggttacctccctggcgcccgggtacc

SEQ ID NO: 14 - DNA, 11801 bp, Artificial Sequence
  pBYe-R1-GFP
  misc_feature (1043)..(1043): any nucleic acid
  misc_feature (1052)..(1052): any nucleic acid
  misc_feature (1078)..(1078): any nucleic acid
cgatcgccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggctatcgttcgtaaatggtgaaaattttcagaaaattgcttttgctttaaaagaaatgatttaaattgctgcaatagaagtagaatgcttgattgcttgagattcgtttgttttgtatatgttgtgttgagaattaattcccctcgactagagtcgagatctggattgagagtgaatatgagactctaattggataccgaggggaatttatggaacgtcagtggagcatttttgacaagaaatatttgctagctgatantgaccttangcgacttttgaacgcgcaataatggnttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaatctagaacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcattttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 15 - DNA, 12509 bp, Artificial Sequence
  pBYe-R2-MRtxG
  misc_feature (1043)..(1043): any nucleic acid
  misc_feature (1052)..(1052): any nucleic acid
  misc_feature (1078)..(1078): any nucleic acid
cgatcgccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggctatcgttcgtaaatggtgaaaattttcagaaaattgcttttgctttaaaagaaatgatttaaattgctgcaatagaagtagaatgcttgattgcttgagattcgtttgttttgtatatgttgtgttgagaattaattcccctcgactagagtcgagatctggattgagagtgaatatgagactctaattggataccgaggggaatttatggaacgtcagtggagcatttttgacaagaaatatttgctagctgatantgaccttangcgacttttgaacgcgcaataatggnttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaatctagaacaatggctaacaaacatctttctttgtctctgttcctggtgcttcttggtctttctgcttctcttgcttctggtcaagttcaacttcaacaacctggtgctgagcttgttaagcctggtgctagtgttaagatgtcttgtaaggctagcggttacaccttcacctcttacaatatgcattgggttaagcagacacctggtagaggtcttgaatggattggtgctatctaccctggtaatggtgatacttcctacaaccagaagttcaagggtaaggctactcttaccgctgataagtcatcttctaccgcttacatgcagctttcttcacttacctctgaggattctgctgtttattactgcgctaggtctacttactacggtggtgattggtacttcaatgtttggggtgctggtactactgttactgtttctgctgcttctactaaggggccctctgtttttcctttggctccttcatctaagtctacctctggtggtactgctgctcttggttgtcttgttaaggattacttccctgagcctgttactgtgtcttggaatagtggtgctcttacttctggtgtgcatacttttccagctgtgcttcaatcttctggtctttactctctttcttctgtggtgactgtgccttcttcttctcttggtactcaaacctacatctgcaacgtgaaccacaagccttctaacaccaaagtggataagaaggctgagcctaagtcttgcgataagactcatacttgtcctccatgtcctgctccagaacttcttggtggtccttctgtttttctgtttccacctaagcctaaggatacccttatgatttctaggactcctgaggttacctgcgttgtggttgatgtttctcatgaagatcctgaggtgaagttcaactggtatgttgatggtgttgaggtgcacaatgctaagactaagcctagagaggaacagtacaactctacttacagggttgtgtctgtgcttactgtgcttcatcaggattggcttaacggtaaagagtacaagtgcaaggtgagcaacaaggctttgcctgctcctattgaaaagaccatctctaaggctaagggtcaacctagagaacctcaagtttacactcttccaccttctagggatgagctgactaagaatcaggtgtcacttacttgcctggtgaagggattttacccttctgatattgctgttgagtgggagtctaatggtcagcctgagaacaattacaagactactcctcctgtgctggattctgatggttcattcttcctgtactctaagctgaccgtggataagtcaagatggcaacagggtaatgtgttctcttgctctgttatgcatgaggctctgcacaatcattacacccagaagtctctgtctctttcacctggtaagtaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtggttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 16 - DNA, 11795 bp, Artificial Sequence
  pBYe-R2-MRtxK
  misc_feature (1043)..(1043): any nucleic acid
  misc_feature (1052)..(1052): any nucleic acid
  misc_feature (1078)..(1078): any nucleic acid
cgatcgccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggctatcgttcgtaaatggtgaaaattttcagaaaattgcttttgctttaaaagaaatgatttaaattgctgcaatagaagtagaatgcttgattgcttgagattcgtttgttttgtatatgttgtgttgagaattaattcccctcgactagagtcgagatctggattgagagtgaatatgagactctaattggataccgaggggaatttatggaacgtcagtggagcatttttgacaagaaatatttgctagctgatantgaccttangcgacttttgaacgcgcaataatggnttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaatctagaacaatggctaacaaacatctttctttgtctctgttcctggtgcttcttggtctttctgcttctcttgcttctggtcagattgtgctttctcagtctcctgctattctgtctgcttctcctggtgaaaaggttacaatgacttgcagggcttcttcttctgtgtcttacattcattggttccagcagaagcctggttcttcacctaagccttggatctacgctacttctaatttggcttctggtgtgcctcttaggttttctggttctggatctggaacctcttactctcttaccatttctagggttgaggctgaagatgctgctacttattattgtcagcagtggacttctaaccctcctacttttggtcagggtactaagcttgagattaagagaactgtggctgctccttccgtgtttattttccctccttctgatgaacaactgaagtctggtactgcttctgttgtgtgccttctgaacaatttctaccctagggaagctaaggtgcagtggaaagttgataatgcactgcagtctggtaactctcaagagtctgttactgagcaggattctaaggatagcacctactcactttcttctacccttaccctgagcaaggctgattatgagaagcacaaggtttacgcttgcgaggttacacatcagggactttcttcacctgtgaccaagtcttttaataggggagagtgctaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtggttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 17 - DNA, 11801 bp, Artificial Sequence
  pBYe-R3-GFP
  misc_feature (1043)..(1043): any nucleic acid
  misc_feature (1052)..(1052): any nucleic acid
  misc_feature (1078)..(1078): any nucleic acid
cgatcgccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggctatcgttcgtaaatggtgaaaattttcagaaaattgcttttgctttaaaagaaatgatttaaattgctgcaatagaagtagaatgcttgattgcttgagattcgtttgttttgtatatgttgtgttgagaattaattcccctcgactagagtcgagatctggattgagagtgaatatgagactctaattggataccgaggggaatttatggaacgtcagtggagcatttttgacaagaaatatttgctagctgatantgaccttangcgacttttgaacgcgcaataatggnttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaatctagaacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtagttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 18 - DNA, 13859 bp, Artificial Sequence
  pBYe3R2K2Mc-BAgD306-6H
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatggctaacaagcacctctcattgtctctcttccttgtgctccttggtctttctgcttctcttgcttctggtaagtacgcattggctgatcctagtcttaaaatggcagatcctaatagattcagaggaaagaatcttccagttttggaccaacttacagatcctccaggagtcaaaagagtttatcatattcaaccttctcttgaggaccctttccaacctcctagcatacctataactgtttattacgctgtgcttgaaagggcttgtaggtctgtgctcctacatgcaccatctgaggctcctcagatagttagaggtgcttctgatgaagcaaggaaacatacttacaatttgaccattgcttggtataggatgggagacaattgtgctatccctattaccgttatggagtacactgaatgtccctacaacaagtctttgggagtttgccctattagaactcagccaaggtggagttactatgattccttctctgcagtttcagaagataatttgggattcttgatgcatgctcctgcctttgagacagctggcacatacttgaggcttgtgaagattaacgactggactgagattacacaattcatacttgaacacagggccagagcatcctgcaagtatgctctcccacttaggattcctccagctgcatgcctcacctcaaaagcttatcaacagggtgtgactgtcgatagtattggcatgttacctcgttttatcccagagaaccagagaaccgttgcattgtactcacttaagatcgcaggatggcacggtccaaagcctccatacacttcaactttgcttccacctgagttgtctgacacaactaacgctacacaacctgaactcgttccagaagacccagaagattctgccctcttagaagatccagctggcactgtgtcttcacaaattcctccaaactggcacattccatcaattcaggatgttgctccacaccatactagtcaccatcaccatcaccattaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccctaaaaccaaaatccagtgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtggttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 19 - DNA, 13263 bp, Artificial Sequence
  pBYe3R2K2Mc-BASP-6H
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatggctaacaagcacctctcattgtctctcttccttgtgctccttggtctttctgcttctcttgcttctggtggagaccaagggcgtgtcatactccttgtgtaccgctgccttcacattcaccaagatcccggctgaaacactccacggaaccgttaccgtggaggtccaatacgccggtacagatggaccttgcaaggttccagctcagatggcggtggacatgcaaactcttaccccagttggaaggttgattaccgctaaccccgttatcactgaaagcactgagaactctaagatgatgttggaacttgatccaccattcggtgactcttacattgtcattggtgtgggagagaagaagatcacccaccactggcacaggagtggtagcactagtcaccatcaccatcaccattaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccctaaaaccaaaatccagtgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtggttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 20 - DNA, 14156 bp, Artificial Sequence
  pBYe3R2K2Mc-BAZsE-6H
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatggctaacaagcacctctcattgtctctcttccttgtgctccttggtctttctgcttctcttgcttctggtatcaggtgcattggagtgagcaacagggactttgtggaaggtatgtcaggtggaacttgggttgatgttgtgttggaacatgggggttgtgtcaccgtgatggcccaggacaaaccgactgtcgacattgagttggttacaacaacggtcagcaacatggccgaggttagatcctactgctatgaggcttcaatttcagacatggctagtgacagccgttgcccaacacaaggtgaagcctaccttgacaagcaatcagacactcaatatgtgtgcaagagaacattggtggacagaggttggggaaacggatgtggacttttcggtaagggaagcctcgtgacatgcgctaaattcgcttgctccaagaagatgaccggaaagagcatccagccagagaacctcgagtaccggattatgttgtcagttcatggttcccagcacagcggaatgatcgttaatgacacaggacatgaaactgatgagaatagagccaaggttgagattacacctaactcaccaagagccgaagccaccctcggaggtttcggaagcttgggacttgattgtgaaccgaggacaggccttgacttttcagatttgtactacttgactatgaataacaagcactggttggttcacaaggaatggttccacgacattccattgccttggcacgctggtgctgacaccggaactccacactggaacaacaaagaggcactcgtggaattcaaggacgcccatgccaagaggcaaactgtcgtggttcttggtactcaagaaggagccgttcacacagcccttgctggtgctctcgaggctgagatggatggtgctaagggaaggctttcctctggccacttgaaatgtcgtttgaagatggataagcttagattgaagggcgtgtcatactccttgtgtaccgctgccttcacattcaccaagatcccggctgaaacactccacggaaccgttaccgtggaggtccaatacgccggtacagatggaccttgcaaggttccagctcagatggcggtggacatgcaaactcttaccccagttggaaggttgattaccgctaaccccgttatcactgaaagcactgagaactctaagatgatgttggaacttgatccaccattcggtgactcttacattgtcattggtgtgggagagaagaagatcacccaccactggcacaggagtggtagcactagtcaccatcaccatcaccattaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccctaaaaccaaaatccagtgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtggttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 21 - DNA, 13828 bp, Artificial Sequence
  pBYe3R2K2Mc-MinV
  misc_feature (1043)..(1043): any nucleic acid
  misc_feature (1052)..(1052): any nucleic acid
  misc_feature (1078)..(1078): any nucleic acid
cgatcgccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggctatcgttcgtaaatggtgaaaattttcagaaaattgcttttgctttaaaagaaatgatttaaattgctgcaatagaagtagaatgcttgattgcttgagattcgtttgttttgtatatgttgtgttgagaattaattcccctcgactagagtcgagatctggattgagagtgaatatgagactctaattggataccgaggggaatttatggaacgtcagtggagcatttttgacaagaaatatttgctagctgatantgaccttangcgacttttgaacgcgcaataatggnttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgaagatggcttcaaacgacgccaatccatcagatggatcagcagcaaatctcgtgccagaagttaacaacgaggtcatggcactagagcctgttgtaggagcagccatcgctgcaccagtggctggacagcaaaatgtgattgatccctggattaggaacaacttcgtgcaggctccaggcggggagtttacagtttctcctaggaacgctcccggagagattctttggtctgctcctcttggaccagatcttaatccttacctttctcatctagcaaggatgtacaatggttatgctggtggattcgaggtgcaagtgattcttgcaggaaatgctttcacagcaggcaagatcattttcgcagccgtccctccaaattttcctacagagggtctaagcccttcccaggtgacaatgtttccccatatcattgttgatgtgagacaacttgagcctgttttgatacctttgccagatgttagaaacaacttctaccattacaatcagtctaatgattctactatcaagctcattgccatgttatatactccattacgtgcaaacaacgctggtgaggatgttttcactgtttcctgcagagttttgacaaggccatctcctgactttgatttcatctttctcgtaccacctacagttgaaagccgtactaagccttttacagtacccatccttacagttgaagagatgactaattctcgatttccaattcctctggagaaattattcacaggtccatctggagctttcgttgttcagccacagaatggcaggtgcactacagacggtgtactgcttggaacaactcagctttccccagtgaacatttgtactttccgaggtgatgtgactcatatcgcaggatctaggaattacaccatgaatttggcttcattgaattggaacaactacgatccaaccgaagagattccagctcctttgggtacaccagatttcgtgggcaagattcagggcgtcctgacccagactactaagggtgacggctcaaccagaggacacaaagctaccgtttatactggtagtgcaccttttactcctaagttgggaagtgtgcagttttcaactgataccgaaaacgacttcgagacacaccaaaacacaaagtttactcctgtgggtgtcatccaggatggatcaactacccacagaaacgagcctcaacagtgggtccttccttcatattcaggtagaaacgtgcataacgttcatcttgctccagctgttgccccaaccttcccaggtgaacaacttctcttcttcagatctactatgcctggatgctctggatatcctaacatggatctcgattgtttgcttcctcaagaatgggtgcagcacttctatcaggaggcagcaccagctcagtccgacgttgcacttctccgtttcgttaacccagacacaggcagggtgttgttcgaatgcaaactacataagtcaggatacgtcactgttgctcacactggtcaacacgatttggtaatcccacctaatggttatttcagatttgactcctgggttaaccagttctataccctggcaccaatgggtaatggcacaggaaggcgtagagcactttaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccctaaaaccaaaatccagtgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtggttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 22 - DNA, 14227 bp, Artificial Sequence
  pBYR2eK2Mc-MinV
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgaagatggcttcaaacgacgccaatccatcagatggatcagcagcaaatctcgtgccagaagttaacaacgaggtcatggcactagagcctgttgtaggagcagccatcgctgcaccagtggctggacagcaaaatgtgattgatccctggattaggaacaacttcgtgcaggctccaggcggggagtttacagtttctcctaggaacgctcccggagagattctttggtctgctcctcttggaccagatcttaatccttacctttctcatctagcaaggatgtacaatggttatgctggtggattcgaggtgcaagtgattcttgcaggaaatgctttcacagcaggcaagatcattttcgcagccgtccctccaaattttcctacagagggtctaagcccttcccaggtgacaatgtttccccatatcattgttgatgtgagacaacttgagcctgttttgatacctttgccagatgttagaaacaacttctaccattacaatcagtctaatgattctactatcaagctcattgccatgttatatactccattacgtgcaaacaacgctggtgaggatgttttcactgtttcctgcagagttttgacaaggccatctcctgactttgatttcatctttctcgtaccacctacagttgaaagccgtactaagccttttacagtacccatccttacagttgaagagatgactaattctcgatttccaattcctctggagaaattattcacaggtccatctggagctttcgttgttcagccacagaatggcaggtgcactacagacggtgtactgcttggaacaactcagctttccccagtgaacatttgtactttccgaggtgatgtgactcatatcgcaggatctaggaattacaccatgaatttggcttcattgaattggaacaactacgatccaaccgaagagattccagctcctttgggtacaccagatttcgtgggcaagattcagggcgtcctgacccagactactaagggtgacggctcaaccagaggacacaaagctaccgtttatactggtagtgcaccttttactcctaagttgggaagtgtgcagttttcaactgataccgaaaacgacttcgagacacaccaaaacacaaagtttactcctgtgggtgtcatccaggatggatcaactacccacagaaacgagcctcaacagtgggtccttccttcatattcaggtagaaacgtgcataacgttcatcttgctccagctgttgccccaaccttcccaggtgaacaacttctcttcttcagatctactatgcctggatgctctggatatcctaacatggatctcgattgtttgcttcctcaagaatgggtgcagcacttctatcaggaggcagcaccagctcagtccgacgttgcacttctccgtttcgttaacccagacacaggcagggtgttgttcgaatgcaaactacataagtcaggatacgtcactgttgctcacactggtcaacacgatttggtaatcccacctaatggttatttcagatttgactcctgggttaaccagttctataccctggcaccaatgggtaatggcacaggaaggcgtagagcactttaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 23 - DNA, 1050 bp, Nicotiana benthamiana
gagctcatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctcatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcatgaattc

SEQ ID NO: 24 - DNA, 453 bp, Nicotiana benthamiana
gagctcactgaggaaatatatagacaaattaagtttggttctatgagttctaatttggacttaagagttgtttgaaattctattttatagtgatgcttataatgtatttggactgttttctgctgtgtgtaagaccttttggtctgtgaactggaaacatacatgaataaatttctttgaatttactggaatttttgcatcaacaaaagaaaaattgaagttactaacttgtaaatggaacaattgtaatgttaaaggatataaatatcttaatatagtgcgatacgaatcacacgaatgcaagactttctctctctgctcccgctcatgctctcggtgcatgttagctaaatatacatcggtgcatccatggcaggagcatgaggacggggatgaggaagggagtgaggagggccaaaagaagtacacatagtttcctttgggagcgaattc

SEQ ID NO: 25 - DNA, 261 bp, Arabidopsis thaliana
gagctcatatgaagatgaagatgaaatatttggtgtgtcaaataaaaagcttgtgtgcttaagtttgtgtttttttcttggcttgttgtgttatgaatttgtggctttttctaatatcaaatgaatgtaagatctcattataatgaataaacaaatgtttctataatccattgtgaatgttttgttggatctcttctgcagcatataactactgtatgtgctatggtatggactatggaatatgattaaagataagaattc

SEQ ID NO: 26 - DNA, 223 bp, Cauliflower mosaic virus
gagctcgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgaattc

SEQ ID NO: 27 - DNA, 26 bp, Artificial Sequence
  primer
gtgagctcgtccgcaaaaatcaccag

SEQ ID NO: 28 - DNA, 30 bp, Artificial Sequence
  primer
cagaattcgtcactggattttggttttagg

SEQ ID NO: 29 - DNA, 283 bp, Agrobacterium tumefaciens
gagctcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgatcggggctgcaggaattc

SEQ ID NO: 30 - DNA, 215 bp, Tobacco mosaic virus
ggtaccggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccagagct

SEQ ID NO: 31 - DNA, 246 bp, Tobacco necrosis virus
ggtaccttgctttcatagatccgtcttcccagagacgttaagaagaagctggagaaaaatattaggttagaagcttgggcgtgacaaacccaagttgcatctcttacgtggttaatcacactgtatgttgacgtacaagccggatcctgggaaacaggtttaacggctcactgtggtggtgggccgtcgatacacttgtatgtgccccaatattggttgtcgagatctctctaggaacccgagctc

SEQ ID NO: 32 - DNA, 982 bp, Solanum tuberosum
gagctcgcccggggatcctctagagtaccctgcaatgtgaccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattattattattattttgatgttcaatgaagttttttttaatttcatatgaagtatacaaaaattcttcatagatttttgtttctatgccgtagttatctttaatatatttgtggttgaagaaatttattgctagaaacgaatggattgtcaatttttttttaaagcaaatatatatgaaattatactgtatattattttagtcatgattaaaatgtggccttaattgaatcatctttctcattcattttttcaaaagcatatcaggatgattgatatttatctattttaaaaattaatttaagggttcaaattaaatttaacttaaaagtgtcctaaccgtagttaaaggtttactttaaaaaaatactatgaaaaatctaatcttctatgaatcgacctgcaggaattc

SEQ ID NO: 33 - DNA, 695 bp, Artificial Sequence
  pUCPMA-M24
gagctcccaattcgccctatagtgagtcgtattacgcgcggagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaagctatcgaattc

SEQ ID NO: 34 - DNA, 743 bp, Nicotiana tabacum
gagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattc

SEQ ID NO: 35 - DNA, 492 bp, Nicotiana tabacum
gagctcaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattc

SEQ ID NO: 36 - DNA, 513 bp, Bean dwarf mosaic virus
gagctctgacaacatcagcaagaacgccctcctagtatattactgttggatgtcagatactatgtcaaaggcatctacttttgtatcgtttgaccttgattatatcggttgattaatgataattgtaataaaaagctattattgaactttcaattcctcaacaaagaaattattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggtttcctttgaattc

(19) United States
(12) Patent Application Publication
(10) Pub. No.: US 2022/0235362 A1
(43) Pub. Date: Jul. 28, 2022

(54) GEMINIVIRAL VECTORS THAT REDUCE CELL DEATH AND ENHANCE EXPRESSION OF BIOPHARMACEUTICAL PROTEINS

(71) Applicant: Arizona Board of Regents on behalf of Arizona State University, Scottsdale, AZ (US)

(72) Inventors: Hugh Mason, Phoenix, AZ (US); Andrew Diamos, Tempe, AZ (US)

(21) Appl. No.: 17/607,843

(22) PCT Filed: Apr. 30, 2020
(86) PCT No.: PCT/US20/30784

§ 371 Date: Oct. 29, 2021

Related U.S. Application Data
(60) Provisional application No. 62/841,098, filed on Apr. 30, 2019.

(51) Int. Cl. C12N 15/82; C12N 1/20; C12N 15/74

(52) U.S. Cl. CPC C12N 15/8205; C12N 1/20; C12N 15/743; C12N 2830/60; C12N 2750/12043; C12N 2750/12022; C12N 2820/60; C12N 15/8258

(57) ABSTRACT

The disclosure relates to a T-DNA binary vector based on bean yellow dwarf virus (BeYDV) that reduces plant cell death and increases transgene expression. In one aspect, the T-DNA region comprise a replicon cassette comprising a rep gene or a repA gene with a mutated translation initiation region. The disclosure also relates to replicating geminiviral expression system based on BeYDV comprising with an expression cassette a sequence encoding Rep and a sequence encoding the promoter of ubiquitin-3 from potato with ubiquitin fusion; an expression cassette comprising a sequence encoding RepA and a sequence encoding the promoter of ubiquitin-3 from potato with ubiquitin fusion; and an expression cassette comprising a promoter region, a 5′ UTR, a sequence encoding a recombinant protein, and a 3′ UTR. These expression cassettes are on different T-DNA cloning vectors or on one T-DNA cloning vector.

### RELATED APPLICATIONS

This application claims the benefit of U.S. provisional patent application 62/841,098, filed Apr. 30, 2019 titled “Geminiviral Vectors That Reduce Cell Death and Enhance Expression of Biopharmaceutical Proteins,” the entirety of the disclosure of which is hereby incorporated by this reference.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 172,147 byte ASCII (text) file named “SeqList” created on Apr. 17, 2020.

### TECHNICAL FIELD

The disclosure relates to replicating geminiviral expression systems modified to reduce cell death while enhancing the production of biopharmaceutical proteins.

### BACKGROUND

Plant-based expression systems offer many potential advantages over traditional systems, including safety, speed, versatility, scalability, and cost (Chen and Davis, 2016; Gleba et al., 2014; Nandi et al., 2016; Tusé et al., 2014). The demonstration that plant-made pharmaceuticals can be glyco-engineered to have authentic human N-glycans, with greater homogeneity and subsequently greater efficacy than their mammalian-produced counterparts further underscores the potential of plant-based systems for the production of therapeutic proteins (Zeitlin et al. 2011, Hiatt et al. 2014, Strasser et al. 2014). Transient expression systems have become the most commonly used systems to produce recombinant proteins in plants (Gleba et al., 2014). However, high accumulation of foreign proteins, especially when ER-targeted, often puts significant stress on the plant cells. In some cases, this may lead to prohibitive levels of tissue necrosis that reduce yields (Hamorsky et al., 2015).

A plant-based transient expression system has been developed which uses the replication machinery from the geminivirus bean yellow dwarf virus (BeYDV) to substantially increase transgene copy number in the plant nucleus, with a subsequent increase in transcription of the target gene (Huang et al., 2009, 2010). This system has been used to produce high levels of vaccine antigens and pharmaceutical proteins in Nicotiana benthamiana leaves (Phoolcharoen et al. 2011; Lai et al. 2012; Moon et al. 2014; Kim et al. 2015; Diamos et al. 2016; Diamos and Mason 2018). High levels of tissue necrosis have been noted when expressing certain proteins using BeYDV vectors, including Ebolavirus glycoprotein, hepatitis B core antigen, GII norovirus particles, monoclonal antibodies and other ER-targeted proteins (Phoolcharoen et al. 2011; Mathew et al. 2014, and unpublished data). Thus, while the BeYDV system can increase the amount of biopharmaceutical protein produced, overall productivity may be reduced or not increased compared to other plant-based expression system due to high level of cell death in the plant. Accordingly, the problem of reducing plant tissue necrosis during the production of biopharmaceutical proteins remains unaddressed.

### SUMMARY

The disclosure relates to a T-DNA region. In certain embodiments, the T-DNA region comprises a replicon cassette and an expression cassette, wherein the replicon cassette comprises a rep gene or repA gene from a mastrevirus with a mutation in its 5′ untranslated region (UTR). In some aspects, the mutation is at the translation initiation site of the rep gene or repA gene, namely at position −3. In certain embodiments, the nucleic acid at position −3 is not A or G, e.g., the nucleic acid at position −3 is T or C. For example, the sequence of the translation initiation site is CACATG. Thus, the disclosure also relates to a T-DNA binary vector having the described T-DNA region.

The disclosure also relates to replicon vector designs. In one aspect, the replicon vector is a T-DNA binary vector with a T-DNA region comprising a sequence encoding RepA and a sequence encoding the promoter of ubiquitin-3 from potato with ubiquitin fusion. In another aspects, the replicon vector is a T-DNA binary vector with a T-DNA region comprising a sequence encoding Rep and a sequence encoding the promoter of ubiquitin-3 from potato with ubiquitin fusion.

The disclosure further relates to a replicating geminiviral expression system. In some embodiments, the replicating geminiviral expression system comprises a first cloning vector with a T-DNA region comprising a sequence encoding Rep and a sequence encoding the promoter of ubiquitin-3 from potato with ubiquitin fusion; a second cloning vector with a T-DNA region comprising a sequence encoding RepA and a sequence encoding the promoter of ubiquitin-3 from potato with ubiquitin fusion; and a third cloning vector with a T-DNA region comprising an expression cassette and no replicon cassette. The expression cassette of the third cloning vector comprises a promoter region, a 5′ UTR, a sequence encoding transgene, and a 3′ UTR. In other embodiments, the replicating geminiviral expression system comprises a T-DNA binary vector comprising a first expression cassette, a second expression cassette, and a third expression cassette. The first expression cassette comprises a sequence encoding Rep and a sequence encoding the promoter of ubiquitin-3 from potato with ubiquitin fusion. The second expression cassette comprises a sequence encoding RepA and a sequence encoding the promoter of ubiquitin-3 from potato with ubiquitin fusion. The third expression cassette comprises a promoter region, a 5′ UTR, a sequence encoding transgene, and a 3′ UTR.

The disclosure is additionally directed to methods of expressing a recombinant protein in plant cell. The methods comprising transforming agrobacteria with the above described T-DNA binary vectors and administering the transformed agrobacteria to a plant cell.

### BRIEF DESCRIPTION OF THE DRAWINGS

FIGS. 1A-1C, in accordance with some embodiments, show controlled expression of Rep and RepA in Nicotiana benthamiana leaves. FIG. 1A depicts a generalized schematic representation of the vectors of the replicating geminiviral expression system based on bean yellow dwarf virus (BeYDV) used in the Examples. RB and LB, the right and left borders of the T-DNA region from Agrobacterium; NOS 3′, the nopaline synthase terminator from Agrobacterium; P19, the RNA silencing suppressor from tomato bushy stunt virus; 35S, the 35S promoter from cauliflower mosaic virus; LIR, the long intergenic region from BeYDV; 5′ UTR, the 5′ untranslated region as described in each experiment; GOI, the gene of interest, as described in each experiment; Ext 3′, the 3′ region from the tobacco extensin gene; SIR, the short intergenic region from BeYDV; Rep/RepA, the replication proteins from BeYDV, which are either present in wildtype form, or are deleted or mutated as described in each experiment.

FIG. 1B depicts a generalized schematic representation of the T-DNA region of the separated Rep/RepA vectors used in the Examples. NPTII, kanamycin resistance cassette; VspB 3′, vegetative storage protein B gene terminator from soybean; Promoter, various promoters as described with 5′ UTR from tobacco etch virus; NOS, the nopaline synthase promoter from Agrobacterium; VspB, the vegetative storage protein B promoter from soybean; Ubi, the ubiquitin-3 promoter from potato; UbiF, Ubi with ubiquitin fusion. FIG. 1C shows that protein expression of results of agroinfiltrated N. benthamiana leaves. Agrobacterium carrying the indicated T-DNA vectors mixed to a final OD of 0.2 for each construct and were infiltrated into the leaves of N. benthamiana. After 4 days post infiltration (DPI), leaf tissue samples were harvested, and protein extracts were analyzed by reducing or nonreducing western blot. In the “Reduced” gel, the lane “35S Rep/35S RepA” was pasted from a different gel than the other lanes (two representative gels of Rep/RepA expression were combined into a single panel). For RT-PCR, RNA was extracted from leaf samples and 50 ng of converted cDNA were PCR amplified with Rep-specific primers.

FIG. 2 depicts, in accordance with some embodiments, replicon accumulation by differential Rep/RepA expression. Leaves of N. benthamiana were agroinfiltrated with either low (UbiF) or high (35S) expression vectors producing combinations of Rep and/or RepA, along with the replicon vector pBY-2e-NVCP. Leaf tissue samples were harvested at 4 days post infiltration (DPI), and 1 μg of extracted total DNA was separated and visualized by ethidium bromide stained agarose gel electrophoresis. The relative intensity of replicon bands was quantified with ImageJ software. Error bars are means±standard deviation of 3 or more independently infiltrated samples.

FIGS. 3A-3B depict, in accordance with some embodiments, NVCP production by differential Rep/RepA expression. Leaves were agroinfiltrated with either low (UbiF) or high (35S) expression vectors producing combinations of Rep and/or RepA, along with the replicon vector pBY-2e-NVCP. FIG. 3A shows the comparison of NVCP production. Leaf tissue samples were harvested at 4-5 DPI, and protein extracts were analyzed for NVCP production by ELISA. Bars represent means±standard deviation from 3 or more independently infiltrated leaf samples. (**) indicates p<0.05 by student's t-test compared to wildtype Rep/RepA. FIG. 3B is a representative leaf imaged at 4-5 DPI under visible light to monitor the development of necrosis.

FIGS. 4A-4B depict, in accordance with some embodiments, exemplary leaves demonstrating Rep/RepA expression induces chlorosis and cell death. For FIG. 4A, leaves were agroinfiltrated with vectors supplying high levels of Rep, RepA, GFP, or an empty vector with coding sequences removed. Leaves were monitored for tissue necrosis, and representative images were taken at 8 DPI. For FIG. 4B, leaves were agroinfiltrated with either Rep/RepA (pRep110) alone, or both pRep110 and the empty replicon vector pBY-EMPTY. Image was taken at 8 DPI.

FIGS. 5A-5C show, in accordance with some embodiments, the expression of GFP and rituximab with modified Rep/RepA vectors. Leaves were coinfiltrated with modified Rep/RepA vectors and replicon vectors expressing either GFP (FIG. 5A) or rituximab (FIG. 5B). For FIG. 5A, the wildtype vector is pBYR2e-GFP, while modified vector is pBYe-R2-GFP. For FIG. 5B, the wildtype vectors for expressing the heavy and light chains are pBYR2e-MRtxG and pBYR2e-MRtxK, while the modified vectors for expressing the heavy and light chains are pBYe-R2-MRtxG and pBYe-R2-MrtxK. For GFP analysis, protein extracts were separated on SDS-PAGE gels, and the GFP band intensity was quantified using ImageJ software. Columns are means±standard deviation of three or more independently infiltrated samples. For rituximab, antibody production was quantified by IgG ELISA. Total soluble protein was determined by Bradford assay using bovine serum albumin and standard. Columns represent means±standard deviation from three or more independently infiltrated leaf samples. (**) indicates p<0.05 by student's t-test compared to wildtype Rep/RepA. FIG. 5C depicts a representative leaf imaged at 4-5 DPI under visible light to monitor the development of necrosis.

FIGS. 6A-6C depict, in accordance with some embodiments, the characterization of Rep/RepA 5′ UTR mutant. Leaves of N. benthamiana were agroinfiltrated with the rituximab-producing replicon vector with (pBYe-R2-MRtx) or without (pBYR2e-MRtx) a mutated Rep/RepA 5′ UTR and analyzed after 4-5 DPI for replicon band intensity quantified from 500 ng total DNA by ethidium bromide stained agarose gel (FIG. 6A) or western blot (inset). FIG. 6B shows the amount of rituximab produced as measured by IgG ELISA. FIG. 6C depicts necrosis of an exemplary leave imaged at 5 DPI.

FIGS. 7A-7D show, in accordance with some embodiments, that replicating vectors require lower Agrobacterium concentration for optimal expression. Leaves of N. benthamiana were agroinfiltrated with the GFP-expressing BeYDV vectors or the nonreplicating vector pEAQ-HT-GFP at the indicated OD600 values. Leaf spots were assayed for GFP production by SDS-PAGE followed by quantification of fluorescence band intensity by ImageJ software (FIG. 7A). Leaf images were taken under UV light (FIG. 7B) or visible light (FIG. 7C). Protein extractions from leaf spots agroinfiltrated at the indicated OD₆₀₀ values with a BeYDV vector expressing an HBc heterodimer were visualized by SDS-PAGE with Coomassie staining (FIG. 7D). Arrow indicates HBc heterodimer band. A representative mock-infiltrated protein extract from a different gel is shown at left for comparison.

FIGS. 8A-8C show, in accordance with some embodiments, virus-derived 5′ and 3′ untranslated regions induce cell death. Leaves of N. benthamiana were agroinfiltrated with pEAQ-HT-GFP, which contains the CPMV 5′ and 3′ UTRs, or the BeYDV GFP vector pBYR2eK2Mc-GFP, at the indicated OD₆₀₀ values and imaged under visible light at 5 DPI (FIG. 8A). Leaves were agroinfiltrated (OD₆₀₀=0.2) with a BeYDV rituximab vectors containing either the NbPsaK 5′ UTR or TMV 5′ UTR and imaged at 5 DPI (FIG. 8B). BeYDV GFP vectors containing the 5′ and 3′ UTRs from tobacco mosaic virus, pea enation mosaic virus, and barley yellow dwarf virus were agroinfiltrated (OD₆₀₀=0.2) and imaged under visible light at 5 DPI (FIG. 8C).

FIG. 9 depicts a comparison of mutations in the 5′ UTR of Rep/RepA on expression of Rep. Leaves were extracted 4 days post-infiltration, and soluble proteins run on SDS-PAGE and western blot probed with rabbit anti-Rep polyclonal serum. WT used vector pBYR2e-GFP; R1 used pBYe-R1-GFP; R2 used pBYe-R2-GFP; R3 used vpBYe-R3-GFP. R1 refers to pBYe-R1-GFP, which has a mutation at −1 (relative to ATG start codon) of the Rep/RepA 5′ UTR (AACATG to AAAATG). R2 refers to pBYe-R2-GFP, which has a mutation at −3 mutation (AACATG to CACATG). R3 refers to pBYe-R3-GFP, which also has a mutation at −3 mutation (AACATG->TACATG).

FIG. 10 depicts a comparison of mutations in the 5′ UTR of Rep/RepA on replicon abundance. DNA was extracted from leaves 4 days post-infiltration, and fractionated on agarose gel, followed by image quantification. WT used vector pBYR2e-GFP; R1 used pBYe-R1-GFP; R2 used pBYe-R2-GFP; R3 used vpBYe-R3-GFP. R1 refers to pBYe-R1-GFP, which has a mutation at −1 (relative to ATG start codon) of the Rep/RepA 5′ UTR (AACATG to AAAATG). R2 refers to pBYe-R2-GFP, which has a mutation at −3 mutation (AACATG to CACATG). R3 refers to pBYe-R3-GFP, which also has a mutation at −3 mutation (AACATG->TACATG). Data are mean+/−SD of three replicated determinations. **, p 0.05.

FIG. 11 depicts a comparison of mutations in the 5′ UTR of Rep/RepA on expression of rituximab in leaves of N. benthamiana co-infiltrated with H and L chain vectors. Leaves were extracted 4 days post-infiltration, and rituximab was assayed in cleared extracts by ELISA. Wildtype used vectors pBYR2e-MRtxG and pBYR2e-MRtxK. R2 Rep used vectors pBYe-R2-MRtxG and pBYe-R2-MRtxK. R3 Rep used vectors pBYe-R3-MRtxG and pBYe-R3-MRtxK. WT used vector pBYR2e-GFP; R1 used pBYe-R1-GFP; R2 used pBYe-R2-GFP; R3 used vpBYe-R3-GFP. R2 constructs have an A→C mutation at −3 mutation (AACATG to CACATG) in the 5′ UTR of Rep/RepA. R3 constructs have an A→T mutation at −3 mutation (AACATG->TACATG) in the 5′ UTR of Rep/RepA. Data are mean+/−SD of three replicate determinations. **, p≤0.05.

FIG. 12 depicts the construct map of pBYe-R1-GFP.

FIG. 13 depicts the construct map of pBYe-R2-MRtxG.

FIG. 14 depicts the construct map of pBYe-R2-MRtxK.

FIG. 15 depicts the construct map of pBYe-R3-GFP.

FIG. 16 depicts the construct map of pBYe3R2K2Mc-BAgD306-6H.

FIG. 17 depicts the construct map of pBYe3R2K2Mc-BASP-6H.

FIG. 18 depicts the construct map of pBYe3R2K2Mc-BAZsE-6H.

FIG. 19 depicts the construct map of pBYe3R2K2Mc-MinV.

FIG. 20 depicts the construct map of pBYR2eK2Mc-MinV.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

As used herein, the terms “bean yellow dwarf virus vector”, “BeYDV vector,” “BeYDV-based vector,” or a vector of the “BeYDV system” comprises all BeYDV sequences, which are the long intergenic region (LIR), the short intergenic region (SIR), and the rep gene or repA gene. In some aspects, the vectors comprise derivative mutants of BeYDV sequences, for example a rep gene or repA gene mutated at its 5′ UTR, namely the sequence 5′ of its translation initiation site.

As used herein, the term “expression cassette” refers to a distinct component of vector DNA, which contains gene sequences and regulatory sequences to be expressed by the transfected cell. An expression cassette comprises four components (listed from 5′ to 3′): a promoter sequence, 5′ untranslated region (5′ UTR), an open reading frame, and a 3′ untranslated region (3′ UTR). The open reading frame includes the portion of a gene spanning the start codon and the stop codon. Thus, the open reading frame comprises a gene sequence. The regulatory sequences are found in the 5′ UTR and the 3′ UTR. The 5′ UTR refers to the sequence from transcription start site to the start codon. In some aspects, the 3′ UTR comprises the 3′ flanking region (also known as the terminator region) of expression cassette. Thus, in certain embodiments, the 3′ UTR comprises the sequence between the stop codon to the poly(A) site, which is part of the gene sequence, and at least one additional terminator sequence.

As used herein, the term “replicon cassette” refers to an expression cassette comprising at least one gene that assists with replication of an organism's DNA sequence. For example, in certain embodiments, the expression vector disclosed herein comprise a replicon cassette comprising the rep gene or repA from BeYDV.

As used herein, the term “replicon vector” refers to a vector that comprises the cis-acting genetic elements necessary to produce replicons. Thus, a replicon vector comprises as its expression cassette a replicon cassette. For example, in certain embodiments, a replicon vector described herein comprises two flanking LIR regions from bean yellow dwarf virus to designate the borders of the replicon. This segment of DNA is amplified via rolling circle replication and other mechanisms by viral and host genes (rep/repA for bean yellow dwarf virus), creating large numbers of DNA copies which serve as transcription templates for the gene of interest in the plant nucleus.

As used herein, the term “terminator” refers to a DNA sequence that contains polyadenylation signals and causes the dissociation of RNA polymerase from DNA and hence terminates transcription of DNA into mRNA. Accordingly, while the term encompasses terminator sequences of known genes, the term also encompasses other sequences that perform the same function, for example, sequences around the short intergenic region of bean yellow dwarf virus.

As used herein, the term “transgene” refers to a gene from one organism that is introduced into another organism.

The disclosure is directed to that modulating the expression of replication genes in a replicating geminiviral expression system based on bean yellow dwarf virus (BeYDV) improves the suitability of such a system to express transgenes in plants, such as for plant production of biopharmaceutical proteins. While extensive work has been done to optimize the gene expression cassette and other aspects of the BeYDV system (Diamos et al., 2016; Diamos and Mason, 2018), vector replication has not been thoroughly investigated.

Geminiviruses are a family of small (˜2.5 kb) single-stranded DNA viruses which replicate in the nucleus of host cells, associating with histones to form viral chromosomes (Pilartz and Jeske, 2003). BeYDV and other mastreviruses produce only four proteins: a coat protein and movement protein, which are produced by the virion sense DNA strand, and two replication proteins, Rep and RepA, produced on the complementary sense DNA strand (C1/C2 genes). Rep and RepA are produced from a single intron-containing transcript: RepA is the predominant protein product from the unspliced transcript, while a relatively uncommon excision of an intron alters the reading frame to produce Rep. Production of all viral proteins is driven by a single bidirectional promoter in the long intergenic region (LIR) which also contains the viral origin of replication. Both divergent transcripts converge at a short intergenic region (SIR), which has bidirectional transcription terminator signals and is suspected to be the origin of complementary strand synthesis (Liu et al., 1998).

Because geminiviruses produce few gene products, they are heavily reliant on host enzymes. The mastrevirus Rep protein, which is produced early in infection, is a multifunctional protein responsible for initiating rolling circle replication by nicking a conserved stem-loop sequence in the LIR. The majority of replication then occurs using cellular machinery to extend the free 3′ end of the nicked viral replicon, though it is likely that Rep recruits many of the involved cellular factors (Gutierrez, 1999). Rep also plays a role in ligating newly synthesized DNA to create circular viral genomes and possesses helicase activity (Choudhury et al., 2006). In the bipartite begomoviruses, Rep has been shown to form homo-oligomers, or possibly hetero-oligomers with RepA or other proteins, which may play a role in replication (Horvath et al., 1998; Krenz et al., 2011).

A primary function of RepA is thought to be the creation of a cellular environment suitable for replication. Some evidence suggests this occurs by binding retinoblastoma-related proteins, which are involved in cell cycle regulation. With RepA bound, previously sequestered transcription factors are able to initiate S-phase gene expression, creating the cellular machinery necessary for viral replication (Gutierrez et al., 2004). An LxCxE motif has been shown to contribute to retinoblastoma-related protein binding (Ruschhaupt et al., 2013). However, other functions of RepA, many of which are still unidentified, have also been shown to enhance viral replication. A set of proteins known as GRAB proteins, which are involved in leaf development and senescence, have also been found to interact with RepA (Lozano-Duran et al., 2011).

Viral proteins are often potent inducers of the plant hypersensitive response, an immune defense mechanism that triggers the release of reactive oxygen species, autophagy, host translation shutoff, and programmed cell death in response to pathogen infection (Dodds and Rathj en, 2010; Zhou et al., 2014; Zorzatto et al., 2015). In the begomoviruses, the bean dwarf mosaic virus nuclear shuttle protein (NSP) was shown to activate the hypersensitive response in bean plants (Garrido-Ramirez et al., 2000), and this activity was mapped to the N-terminus of the NSP (Zhou et al., 2007). As a countermeasure, the TrAP protein from tomato leaf curl New Delhi virus prevents the activation of the hypersensitive response generated by its NSP (Hussain et al., 2007). Additionally, the NSP is known to interact with a host immune NB-LRR receptor-like kinase to enhance virus pathogenicity and is involved in preventing translation shutoff in response to virus infection (Sakamoto et al., 2012; Zhou et al., 2014). The Rep protein from African cassava mosaic virus also elicited the hypersensitive response in Nicotiana benthamiana (van Wezel et al., 2002), and it was further reported that altering a single amino acid reversed hypersensitive response induction without affecting protein function (Jin et al., 2008). While many studies have focused on the begomoviruses, the role of the hypersensitive response during mastrevirus infection has not been investigated.

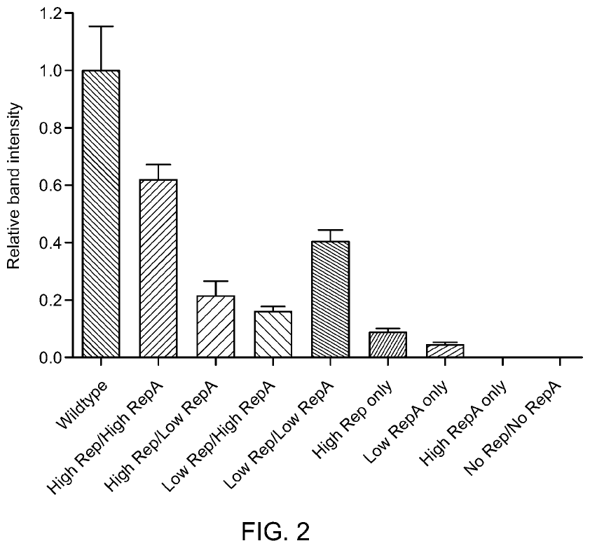

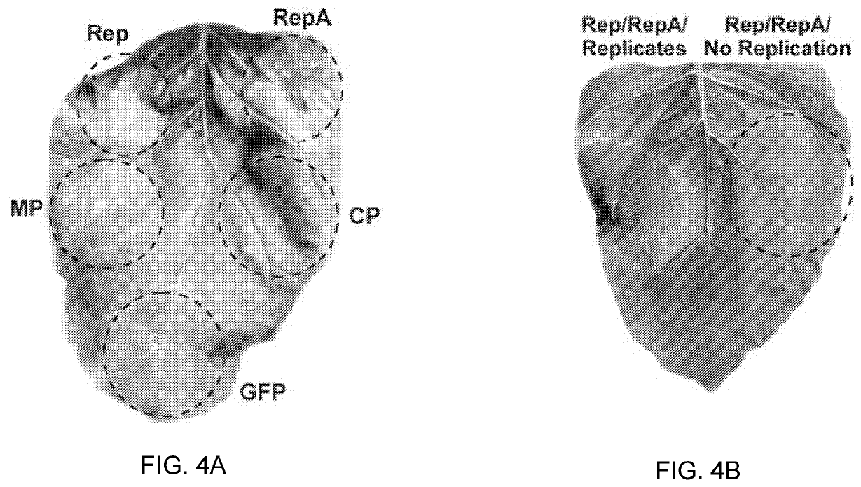

As shown in the Examples, by reducing expression of Rep and RepA, BeYDV-based expression vectors elicit lower levels of cell death. The reduced level of cell death does not come as the cost of transgene expression. In fact, the reduced levels of cell death results in a corresponding increase in the production of vaccine antigens and monoclonal antibodies (see, for example, FIG. 3, FIGS. 5B, and 5C).

In some embodiments, the disclosure is directed to a T-DNA region design, wherein the T-DNA region comprises a replicon cassette and an expression cassette, wherein the replicon cassette comprises a rep gene or repA gene from a mastrevirus that has a mutation in the initiation site at position −3, and the nucleic acid at position −3 is not A or G. For example, the nucleic acid at position −3 is T or C. In certain embodiments, the initiation site sequence of the mutated rep gene or repA gene is CACATG. In other embodiments, the initiation site sequence of the mutated rep gene or repA gene is TACATG. In some embodiments, the rep gene or the repA gene is from bean yellow dwarf virus. In some aspects, the nucleic acid sequence of the repA gene has at least 80% similarity, at least 85% similarity, at least 90% similarity, at least 95% similarity, at least 97% similarity, at least 98% similarity, or at least 99% similarity with the sequence spanning position 1308 to 2398 of GeneBank Y11023.2. In some aspects, the nucleic acid sequence of the rep gene has at least 80% similarity, at least 85% similarity, at least 90% similarity, at least 95% similarity, at least 97% similarity, at least 98% similarity, or at least 99% similarity with the sequence spanning position 1308 to 1519 of GeneBank Y11023.2.

To further enhance expression of expression cassette (which comprises a promoter region, a 5′ untranslated region (UTR), a sequence encoding transgene; and a 3′ UTR), the 5′ UTR and/or the 3′ UTR of the expression cassette may be selected from 5′ UTRs and 3′ UTRs that have been identified to result in enhanced recombinant protein expression in plants (see PCT/US2019/020621, the contents of which are incorporated by reference herein). The 3′ UTR regions that provide enhanced production of the recombinant protein include the extensin 3′ UTR (also referenced herein as the extensin terminator), N. benthamiana actin 3′ UTR (NbACT3), potato proteinase inhibitor II 3′ UTR (Pin2), bean dwarf mosaic virus DNA B nuclear shuttle protein 3′ UTR (BDB), N. benthamiana 18.8 kDa class II heat shock protein 3′ UTR (NbHSP), pea rubisco small subunit 3′ UTR (RbcS), A. thaliana heat shock protein 3′ UTR (AtHSP), cauliflower mosaic virus 35S 3′ UTR (35S), and Agrobacterium nopaline synthase 3′ UTR (NOS). The sequences of these 3′UTR are well-known in the art.

In some aspects, the nucleic acid sequence of the extensin terminator is selected from the terminator sequences of the extensin gene in Nicotiana tabacum, Nicotiana tomentosiformis, Nicotiana plumbaginifolia, Nicotinana attenuata, Nicotinana sylvestris, Nicotiana benthamiana, Solanum tuberosum, Solanum lycopersicum, Solanum pennellii, Capsicum annuum, and Arabidopsis thaliana, the sequences of which are determinable from GenBank or the Sol Genomics Network. The nucleic acid sequence of the extension terminator comprises a polypurine sequence, an atypical near upstream element (NUE), an alternative polyA site, a far upstream element (FUE)-like region, a major NUE, and a major polyA region, and in certain embodiments, the nucleic acid sequence has at least 72%, at least 73%, at least 74%, at least 75%, at least 76%, at least 77%, at least 78%, at least 79%, 72%, 73%, 74%, 75%, 76%, 77%, 78%, or 79% identity to the sequence of the tobacco (N. tabacum) extension terminator. In some embodiments, the nucleic acid sequence of the extension terminator is that of the tobacco extensin gene. In certain embodiments, the portion of the extensin 3′ UTR in the disclosed vector lacks the intron. In a particular embodiment, the 3′ UTR region of the vector comprises an intronless tobacco extensin terminator (EU). Thus in some aspects, the nucleic acid sequence of EU spans nt 2764-3126 of the complete N. tabcacum gene for extensin (GenBank D13951.1). In certain other embodiments, the disclosed vector comprises intron-containing extensin terminator. Thus in some aspects, the 3′ UTR region of the vector comprises an intron-containing tobacco extensin terminator (IEU). In such embodiments, the nucleic acid sequence of IEU spans nt 2396-3126 of the complete N. tabcacum gene for extensin (GenBank D13951.1).

In some aspects, the nucleic acid sequence of NbACT3 comprises nt 1460-1853 of actin gene (Gene ID Niben101Scf00096g04015.1). In some aspects, the nucleic acid sequence of NbACT3 comprises nt 33-1023 of the sequence set forth in SEQ ID NO. 23. In some aspects, the N. benthamiana actin 3′ UTR is not the entirety of the 3′ UTR, but only the downstream 617-nt region of NbACT3 (NbACT617). In such embodiments, the nucleic acid sequence of NbACT617 comprises nt 606-1023 of the sequence set forth in SEQ ID NO. 23. In other aspects, the N. benthamiana actin 3′ UTR is not the entirety of the 3′ UTR, but only the downstream 567-nt region of NbACT3 (NbACT567).

In some embodiments, the nucleic acid sequence of Pin2 spans nt 1507-1914 of the potato gene for proteinase inhibitor II (GenBank: X04118.1). In some aspects, the sequence of pinII is obtained from pHB114 (Richter et al., 2000) by SacI-EcoRI digestion.

In some embodiments, the nucleic acid sequence of BDB comprises the 3′ end of the nuclear shuttle protein, the intergenic region, the 3′ end of the movement protein, and additional 200 nt downstream of the movement protein sequence (BDB501), which spans nt 1213-1713 of bean dwarf mosaic virus segment DNA-B (GenBank: M88180.1). In some embodiments, the nucleic acid sequence of BDB comprises only the 282 nucleotides that include the 3′ end of the nuclear shuttle protein, the intergenic region, and the 3′ end of the movement protein (BDB282).

In some embodiments, the nucleic acid sequence of NbHSP comprises the complement to nt 988867-989307 of the sequence of Gene ID Niben101Scf04040. In some aspects, the nucleic acid sequence of NbHSP spans nt 33-424, nt 33-447, nt 33-421, nt 33-453, nt 45-424, nt 45-447, nt 45-421, or nt 45-453 of the sequence set forth in SEQ ID NO. 24. In one embodiment, the nucleic acid sequence spanning nt 45-421 of the sequence set forth in SEQ ID NO. 24 is NbHSP. In embodiments, the nucleic acid sequence of NbHSPb comprises the complement to nt 988942-989307 of the sequence of Gene ID Niben101Scf04040. In some aspects, the nucleic acid sequence spanning nt 45-372 of the sequence set forth in SEQ ID NO. 24 is NbHSPb.

In some embodiments, the nucleic acid sequence of rbcS comprises a sequence that is complementary to the sequence spanning nt 6-648 of transient gene expression vector pUCPMA-M24 (GenBank: KT388099.1). In some aspects, the sequence of rbcS is obtained from pRTL2-GUS (Carrington et al., 1999) by SacI-EcoRI digestion.

In some embodiments, the nucleic acid sequence of AtHSP comprises nt 1-250 of the partial sequence of the A. thaliana heat shock protein 18.3 gene (GenBank KP008108.1). In some aspects, the nucleic acid sequence of AtHSP spans nt 7-257 of SEQ ID NO. 25.

In some embodiments, the nucleic acid sequence of 35S comprises a sequence spanning nt 3511-3722 of plant transformation vector pSITEII-8C1 (GenBank: GU734659.1). In some aspects, the sequence of 35S is set forth in nt 7-218 of SEQ ID NO. 26. In some aspects, the sequence of 35S is the sequence of the amplification of pRTL2-GUS (Carrington et al 1991) using the primers 35STm-1 (SEQ ID NO. 27) and 35STm-2 (SEQ ID NO. 27).

In some embodiments, the nucleic acid sequence of NOS comprises nt 22206-22271 of the T-DNA region of cloning vector pSLJ8313 (GenBank: Y18556.1). In some aspects, the sequence of NOS is that of the fragment obtained from pHB103 (Richter et al., 2000) by SacI-EcoRI digestion. In some aspects, the nucleic acid sequence of NOS is set forth in nt 6-261 of SEQ ID NO. 29.

In some embodiments, the 3′ UTR region comprises at least one member from the group consisting of: EU5, IEU, NbACT3, NbACT617, NbACT567, Pin2, BDB501, BDB282, NbHSP, NbHSPb, RbcS, AtHSP, 35S, and NOS. In certain embodiments, the 3′ UTR region of the vector consists of a terminator selected from the group consisting of: EU, NbACT3, Pin2, BDB501, NbHSP, RbcS, NbACT617, NbACT567, NbHSPb, and AtHSP. In some implementations, the 3′ UTR region of the vector consists of a terminator selected from the group consisting of: EU, NbACT3, Pin2, BDB501, NbHSP, and RbcS.

In some aspects, the 3′ UTR comprises two terminators, which produces a double terminator. The double terminator may be a repeat of same terminator or a combination of different terminators (for example, a fusion of two different terminators). In some embodiments, the double terminator consists of EU with NbACT, P19, NbHSP, SIR, NOS, 35S, tobacco mosaic virus 3′ UTR (TMV), BDB501, tobacco necrosis virus-D 3′ UTR (TNVD), pea enation mosaic virus 3′ UTR (PEMV), or barley yellow dwarf virus 3′ UTR (BYDV). In some aspects, the aforementioned pair of terminators are arranged where EU is arranged upstream of the other terminator, which is denoted as EU+NbACT, EU+P19, EU+NbHSP, EU+SIR, EU+NOS, EU+35S, EU+TMV, EU+BDB501, EU+TNVD, EU+PEMV, or EU+BYDV. In some embodiments, the double terminator consists of 35S with NbACT3, NOS, EU, NbHSP, Pin2, or BDB501. In some aspects, the aforementioned pair of terminators are arranged where 35S is arranged upstream of the other terminator, which is denoted as 35S+NbACT3, 35S+NOS, 35S+EU, 35S+NbHSP, 35S+Pin2, or 35S+BDB501. In some embodiments, the double terminator consists of IEU with SIR, 35S, or LIR. In some aspects, the aforementioned pair of terminators are arranged where IEU is arranged upstream of the other terminator, which are denoted as IEU+SIR, IEU+35S, or IEU+LIR. In some embodiments, the double terminator consists of NbHSP with NbACT3, NOS, or Pin2. In some aspects, the aforementioned pair of terminators are arranged where NbHSP is upstream of the other terminator, which is denoted as NbHSP+NbACt3, NbHSP+NOS, or NbHSP+Pin2. In some embodiments, the double terminator consists of NOS with 35S, where NOS is arranged upstream of 35S (NOS+35S).

As used herein, the term “P19” refers to the P19 suppressor of RNAi silencing. An exemplary vector backbone that comprises P19 is pEAQ-HT (see Sainsbury et al., 2009).

In accordance with certain embodiments, the nucleic acid sequence of TMV spans nt 489-693 of the tobacco mosaic virus isolate TMV-JGL coat protein gene (GenBank: KJ624633.1). In some aspects, the nucleic acid sequence of TMV is set forth in nt 7-211 of SEQ ID NO. 30.

In accordance with certain embodiments, the nucleic acid sequence of TNVD has at least 85% identity, preferably 87% identity, to the sequence spanning nt 3457-3673 of the complete genome of tobacco necrosis virus D genome RNA (GenBank: D00942.1). In other embodiments, the nucleic acid sequence of TNVD has at least 90%, preferably 93%, sequence identity with nt 3460-3673 of tobacco necrosis virus-D genome (GenBank: U62546.1). In some embodiments, the nucleic acid sequence of TNVD comprises the sequence set forth in nt 29-222 of SEQ ID NO. 31.

In accordance with certain embodiments, the nucleic acid sequence of PEMV has at least 95%, preferably 98%, sequence identity with nt 3550-4250 of the pea enation mosaic virus-2 strain UK RNA-dependent RNA-polymerase, hypothetical protein, phloem RNA movement protein, and cell-to-cell RNA movement protein genes (GenBank: AY714213.1). In some aspects, the nucleic acid sequence of PEMV is set forth in nt 1-703 of SEQ ID NO. 13.

In accordance with certain embodiments, the nucleic acid sequence of BYDV has at least 95%, preferably 99%, sequence identity with nt 4807-5677 of barley yellow dwarf virus—PAV genomic RNA (GenBank: X07653.1). In some aspects, the nucleic acid sequence of BYDV is set forth in nt 5-875 of SEQ ID NO. 11.

SEQ ID NOs. 23-36 provides the nucleic acid sequences for incorporating the aforementioned 3′ UTRs into the T-DNA region. The nucleic acid sequence of the template for incorporating NOS is set forth in SEQ ID NO. 29. The nucleic acid sequence of the template for incorporating 35S is set forth in SEQ ID NO. 26. The nucleic acid sequence of the template for incorporating pinII is set forth in SEQ ID NO. 32. The nucleic acid sequence of the template for rbcS is set forth in SEQ ID NO. 33. The nucleic acid sequence of the template for incorporating IEU is set forth in SEQ ID NO. 34. The nucleic acid sequence of the template for incorporating EU is set forth in SEQ ID NO. 35. The nucleic acid sequence of the template for incorporating NbHSP is set forth in SEQ ID NO. 24. The nucleic acid sequence of the template for incorporating NbACT3 is set forth in SEQ ID NO. 23. The nucleic acid sequence of the template for incorporating BDB501 is set form in SEQ ID NO. 36. The nucleic acid sequence of the template for incorporating AtHSP is set forth in SEQ ID NO. 25. The nucleic acid sequence of the template for incorporating barley yellow dwarf virus's (BYDV's) 3′ UTR is set forth in SEQ ID NO. 11. The nucleic acid sequence of the template for incorporating TNVD 3′ UTR is set forth in SEQ ID NO. 31. The nucleic acid sequence of the template for incorporating PEMV 3′ UTR is set forth in SEQ ID NO. 13. The nucleic acid sequence of the template for incorporating tobacco mosaic virus 3′ UTR is set forth in SEQ ID NO. 30.

In some embodiments, the 5′ UTR comprises the 5′ UTR of native Nicotiana benthamiana NbPsaK, the 5′ UTR from barley yellow mosaic virus, or the 5′ UTR from cowpea mosaic virus. In some aspects, the 3′ UTR comprises the 3′ UTR from barley yellow mosaic virus or the 3′ UTR from cowpea mosaic virus. In certain implementations where the 5′ UTR and the 3′ UTR of the expression cassette is from a virus, the 5′ UTR and the 3′ UTR should come from the same virus, for example if the virus is pea enation mosaic virus. In certain embodiments, the 5′ UTR of the expression cassette does not comprise the 5′ UTR from tobacco mosaic virus or the 5′ UTR from pea enation mosaic virus. In certain embodiments, the 3′ UTR does not comprise the 3′ UTR from pea enation mosaic virus.

The expression level of the expression cassette may also be further enhanced by the selection of a strong promoter, for example, 35S promoter from cauliflower mosaic virus.

In a particular embodiment, the T-DNA region design comprises PinII 3′ UTR, P19, 35S promoter, LIR, NbPsaK truncated 5′ UTR, the transgene, intronless extensin 3′ UTR, NbAct3 3′ UTR, Rb7 MAR, SIR, and Rep/RepA with mutated 5′ UTR. In some aspects, the arrangement of the T-DNA region from 5′ to 3′ is: PinII 3′ UTR-P19-35S promoter-LIR-35S promoter-NbPsaK truncated 5′ UTR-transgene-intronless extensin 3′ UTR-NbAct3 3′ UTR-Rb7 MAR-SIR-Rep/RepA with mutated 5′ UTR-LIR.

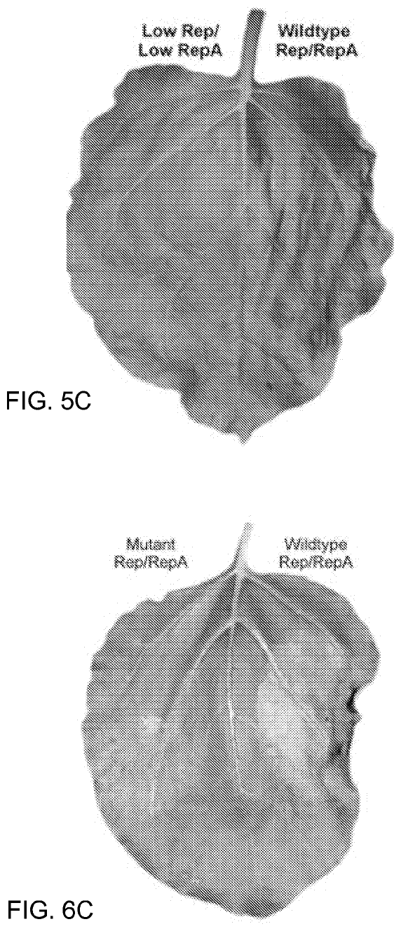

For the production of recombinant proteins with DNA-based systems, the development of cell death depends on the individual composition of the protein being produced, subcellular localization of the target protein (Howell, 2013), glycosylation of the target protein (Hamorsky et al., 2015), target protein expression level, Agrobacterium strain (Diamos et al., 2016) and concentration (Wroblewski et al. 2005, FIG. 7D), DNA elements like matrix attachment regions (Diamos et al., 2016), 5′ and 3′ UTR elements (FIG. 7C/7E), viral replication elements (FIG. 3B/4B/5C), and plant health and growth conditions (Matsuda et al., 2017; Qian et al., 2016). Modifying these factors allows enhanced accumulation of proteins that may, under less favorable conditions, elicit a cell death response. Though the mechanism by which the Rb7 MAR reduces cell death in this system is unknown, larger replicons accumulate to lower amounts than smaller replicons, and thus incorporation of the long 1.2 kb Rb7 MAR can also reduce replicon accumulation. The optimal combination of factors varies depending on the transgene of interest. The optimal level of Rep/RepA expression also can vary depending on the toxicity of the transgene of interest. These modifications will allow high-level production of otherwise toxic biopharmaceutical proteins.

In some embodiments, the element of the replicon cassette may be separated from the elements for expression of the transgene, for example a replicating geminiviral expression system comprising three cloning vectors. One of the cloning vectors comprises a T-DNA region that lacks a replicon cassette but comprises an expression cassette that corresponds to above described expression cassette. The other two cloning vectors are replicon vectors where its T-DNA region comprises a sequence encoding Rep or RepA. In some aspects, the T-DNA region of the replicon vectors further comprise a sequence encoding the promoter of ubiquitin-3 from potato with ubiquitin fusion. In such embodiments, the promoter of ubiquitin-3 from potato with ubiquitin fusion drives the expression of Rep and RepA. As the optimal expression level of Rep/RepA varies depending on the gene of interest, the replicon vector may comprise other promoter regions to drive the expression of Rep and RepA. In some aspects, the promoter driving the expression of Rep in one replicon vector is different than the promoter driving the expression of RepA in the other replicon vector. However, in particular embodiments, the ratio of Rep expression to RepA expression is kept at 1:1.

To reduce the amount of agrobacteria needed for infiltration of plant, the three cloning vector replicating geminiviral expression system can readily be simplified into a single vector that supplies all three expression cassettes from a single T-DNA plasmid. In such non-limiting embodiments, the T-DNA binary vector comprising three expression cassettes wherein each of the expression cassette comprises the elements of the above described cloning vectors. For example, one of the expression cassettes comprises a sequence encoding Rep and a sequence encoding the promoter of ubiquitin-3 from potato with ubiquitin fusion, while another one of the expression cassettes comprises a sequence encoding RepA and a sequence encoding the promoter of ubiquitin-3 from potato with ubiquitin fusion. These two expression cassettes correspond to the two replicon vectors. The third expression cassette comprises a promoter region, a 5′ UTR; a sequence encoding a transgene; and a 3′ UTR.

Also disclosed are methods of expressing a recombinant protein in plant cell using the above described T-DNA region design, T-DNA binary vectors, and replicating geminiviral expression system.

In some embodiments, the method comprises administering to a plant cell a composition comprising a first transformed Agrobacterium, a second transformed Agrobacterium, and a third Agrobacterium. The first transformed Agrobacterium is transformed with a first T-DNA binary vector, and the T-DNA region of the first T-DNA binary vector comprises a sequence encoding Rep and a sequence encoding the promoter of ubiquitin-3 from potato with ubiquitin fusion. The second transformed Agrobacterium is transformed with a second T-DNA binary vector, and the T-DNA region of the second T-DNA binary vector comprises a sequence encoding RepA and a sequence encoding the promoter of ubiquitin-3 from potato with ubiquitin fusion. The third transformed Agrobacterium is transformed with a third T-DNA binary vector, and the T-DNA region of the third T-DNA binary vector comprises an expression cassette and no replicon cassette. The expression cassette comprises a promoter region, a 5′ UTR, a sequence encoding the recombinant protein; and a 3′ UTR.

In certain embodiments, the method comprises administering to a plant cell a composition comprising transformed Agrobacterium, wherein the transformed Agrobacterium is transformed with a T-DNA binary vector having a T-DNA region comprising an expression cassette comprising a sequence encoding the recombinant protein and a replicon cassette comprising a mutated rep gene or repA gene. The mutated rep gene or repA gene comprises a mutation in its 5′ UTR. In some aspects, the mutation is in the initiation site sequence, and the initiation site sequence of the mutated Rep/RepA gene is CACATG. In other aspects, the mutation is in the initiation site sequence, and the initiation site sequence of the mutated Rep/RepA gene is TACATG.

In still other non-limiting embodiments, the method comprises administering to a plant cell a composition comprising transformed Agrobacterium, wherein the transformed Agrobacterium is transformed with a T-DNA binary vector having a T-DNA region comprising three expression cassettes. One expression cassette comprises a sequence encoding Rep and a sequence encoding the promoter of ubiquitin-3 from potato with ubiquitin fusion. Another expression cassette comprises a sequence encoding RepA and a sequence encoding the promoter of ubiquitin-3 from potato with ubiquitin fusion. And the third expression cassette comprises a promoter region, a 5′ UTR, a sequence encoding the recombinant protein; and a 3′ UTR.

### Illustrative, Non-Limiting Example in Accordance with Certain Embodiments

The disclosure is further illustrated by the following examples that should not be construed as limiting. The contents of all references, patents, and published patent applications cited throughout this application, as well as the Figures, are incorporated herein by reference in their entirety for all purposes.

### 1. Controlled Production of Rep and RepA in Plant Leaves

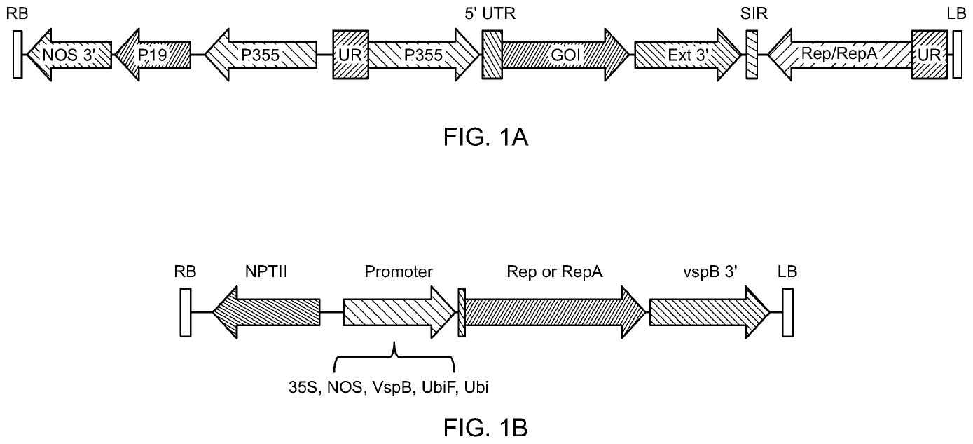

In the BeYDV expression system (FIG. 1A), production of Rep/RepA leads to excision, circularization, and replication of any gene expression cassette flanked by the cis-acting LIRs. A Rep/RepA-supplying vector could be delivered in trans to amplify a replication-deficient BeYDV containing the LIRs but lacking Rep/RepA (Huang et al., 2009). However, this system was only capable of producing Rep and RepA together, at constant high levels under the control of the strong 35S promoter from cauliflower mosaic virus. To study replication, a modular system was created using promoters of varying strengths to express Rep and RepA at controlled levels.

To create a modular system to study vector replication, a series of Agrobacterium T-DNA expression vectors were constructed that separately expressed either Rep or RepA under the control of five different promoters: the 35S promoter, the nopaline synthase promoter from Agrobacterium (NOS), the vegetative storage protein B promoter from soybean (vspB), or the ubiquitin-3 promoter from potato with (UbiF) or without (Ubi) ubiquitin fusion (FIG. 1B). To characterize the expression of Rep and RepA by these vectors, they were infiltrated into the leaves of N. benthamiana and analyzed by western blot and RT-PCR. Rep and RepA from the related wheat dwarf virus are known to form oligomeric complexes (Missich et al., 2000). Antibodies targeting both Rep and RepA produced together in their native wildtype configuration reacted strongly with nonreduced protein extracts, revealing large complexes near 250 kDa in size. RepA produced two distinct high molecular weight bands, whereas Rep produced only a single resolvable band (FIG. 1C, nonreduced). However, when Rep and RepA were expressed together, only a single band at the size of rep alone was observed (FIG. 1C, right panel). Under reducing conditions, Rep (predicted 39 kDa) produced predominately monomeric 35-40 kDa bands, while RepA (predicted 33 kDa) showed 65-75 kDa bands suggestive of oligomeric forms. Interestingly, when both Rep and RepA were coexpressed, a slightly larger 45-50 kDa band of unknown origin also appeared (FIG. 1C). RT-PCR and western analysis both showed that the 35S construct far exceeded the other expression vectors, followed by the NOS, vspB, UbiF constructs, with the unfused Ubi construct providing the weakest expression (FIG. 1C).

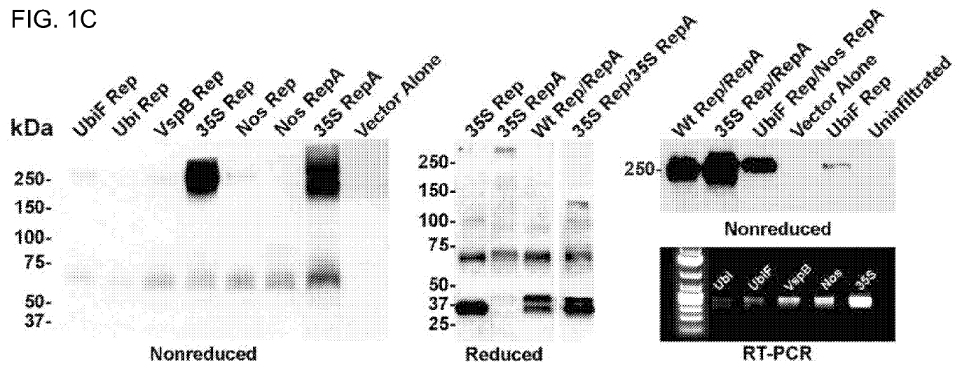

While the 35S promoter is widely known to drive high levels of gene expression, the NOS promoter was reported to be 30-fold weaker than the 35S in transgenic plants (Sanders et al., 1987). All other promoters tested produced substantially lower Rep/RepA than 35S (FIG. 1C); however these levels were still able to provide robust accumulation of viral replicons (FIG. 2) that were present in high enough quantities to be readily visible on ethidium bromide stained gels (data not shown). The potato Ubi3 promoter has been reported to have 5- to 10-fold increase in activity when a reporter gene was translationally fused to ubiquitin (Garbarino and Belknap, 1994). As shown in FIG. 1C, translational fusion of Rep to ubiquitin enhanced its accumulation. As geminiviruses encode few proteins, they rely heavily on host enzymes for replication. The mastrevirus wheat dwarf virus RepA preferentially forms octamers while Rep forms 6-8 subunit oligomers, which assemble at the initiation site and are thought to recruit host replication machinery (Gutierrez et al., 2004). Among the begomoviruses, tomato yellow leaf curl Sardinia virus Rep was found to form dodecamers with helicase activity (Clerot and Bernardi, 2006), and the self-interaction of Abutilon mosaic virus Rep was demonstrated in planta (Krenz et al., 2011). Inventors found BeYDV Rep and RepA form high molecular weight bands consistent with the formation of oligomers comprised of 6-8 monomers (FIG. 1C).

### 2. Impact of Rep and RepA Ratio on Efficiency of BeYDV Replications

To determine the effects of altered Rep and RepA expression on replicon amplification, a replicon vector pBY-2e-sNV encoding a synthetic GI norovirus capsid protein (NVCP) was coinfiltrated with Rep and RepA supplying vectors. For simplicity, further experiments were performed with either UbiF vectors for low expression or 35S vectors for high expression, as no major notable differences were observed among the lower expressing constructs. The vector pBYR2e-sNV, which contains the wildtype Rep/RepA configuration driven by the native LIR promoter, was used as a control. In agreement with previous data on mastrevirus replication (Huang et al., 2009; Ruschhaupt et al., 2013), no replication was detected when RepA alone was supplied, and very low replication was detected when Rep was supplied alone with either a weak or strong promoter (FIG. 2). However, coinfiltration of both Rep and RepA resulted in robust replication (FIG. 2). Interestingly, overproduction of either Rep or RepA relative to the other resulted in impaired replication, suggesting that the relative abundance of each protein is important for efficient replication (FIG. 2). Although expression of Rep and RepA by the strong 35S promoter was comparable to or exceeded wildtype expression levels (FIG. 1C), the wildtype configuration resulted in a consistent increase in replicon accumulation, possibly due to differing of ratios of Rep/RepA expression (FIG. 2). These results show that the level of vector replication can be controlled by differential expression of Rep and RepA.

There is discrepancy in the necessity of RepA for mastreviral rolling circle replication. In cell culture experiments with wheat dwarf virus (Collin et al., 1996) or BeYDV (Hefferon, 2003; Liu et al., 1998), intron-deleted rep has been reported to support high levels of replication. In contrast, maize streak virus only supported very low levels of replication in the absence of RepA (Ruschhaupt et al., 2013). In agreement with the results of Ruschhapt et al, only low levels of replication was observed when expressing rep alone in N. benthamiana leaves, even in the presence of high levels of Rep (FIG. 2). Despite the small increase in NVCP-expressing replicon accumulation by supplying Rep alone, a small decrease in NVCP expression was observed, perhaps indicating that replicons generated this way are less available for transcription, or that some other function of RepA increases transgene expression. Notably, expression of RepA alone also had a small negative effect on NVCP expression, indicating that both Rep and RepA are indeed required for productive enhancement of transgene expression (FIG. 3A). Furthermore, the relative ratio of Rep and RepA is essential for replication. Expression of both Rep and RepA from relatively weak promoters still resulted in robust replicon production, but this did not occur if either Rep or RepA were overexpressed relative to the other (FIG. 3). Rep and RepA share the same N-terminus, including DNA binding and oligomerization domains, which may permit hetero-oligomerization (Horvath et al., 1998; Missich et al., 2000). Proper hetero-oligomerization of Rep and RepA may be disrupted when either monomer is overexpressed relative to the other.

In their native configuration, production of either Rep or RepA is controlled by the excision of an intron and thus the frequency of intron removal controls the relative abundance of each protein. For maize streak virus in infected maize, it has been reported that approximately 80% of transcripts produce RepA, and only 20% produce Rep (Wright et al., 1997). 35S-driven Rep and RepA produced as much or more combined Rep/RepA than the wildtype gene (FIG. 1C), yet had reduced replicon amplification (FIG. 2). By reducing western blot it was possible to distinguish the 39 kDa Rep, which forms a single ˜35-40 kDa band when expressed alone, from the 33 kDa RepA, which ran as a 65-75 kDa band when expressed alone, perhaps suggestive of dimer formation (FIG. 1C). 35S-driven Rep/RepA consistently overproduced the Rep monomer-sized band and underproduced the RepA dimer-sized band compared to the wildtype configuration (FIG. 1C), which suggests that 35S-driven Rep/RepA may not produce the proper ratio of each protein, thereby leading to reduced replication.

### 3. Impact of Reducing Vector Replication on Cell Death and Transgene Expression

Previously, it was shown that coinfiltration of a replicon vector and a Rep/RepA-supplying vector encoding both Rep and RepA together in the native configuration enhances the production of target proteins (Huang et al., 2009; Mor et al., 2003). To further characterize the relationship between replicon amplification and target protein accumulation, the production of NVCP from replicons amplified with variable levels of Rep and RepA was measured by ELISA. The control vector psNV120e contains no BeYDV elements and thus cannot replicate, whereas pBY-2e-sNV contains the intergenic regions from BeYDV necessary for replication. Interestingly, even in the absence of Rep and RepA, pBY-2e-sNV substantially increased NVCP expression by 3.1-fold compared to psNV120e, accumulating NVCP at 0.57 mg/g LFW (FIG. 3A). NVCP expression was further enhanced by an additional 2.7-fold when pBY-2e-sNV was coinfiltrated with 35S-driven Rep/RepA or when Rep/RepA were supplied by the wildtype LIR promoter, yielding NVCP at approximately 1.5 mg/g LFW (FIG. 3A). Unexpectedly, coinfiltration with vectors supplying Rep and RepA at lower than wildtype levels produced the highest yield of NVCP, reaching 2.0 mg/g LFW. The increase in NVCP expression was notably associated with a reduction in plant cell death (FIG. 3B). Among replicating vectors, NVCP expression was lowest when the production of either Rep or RepA was substantially higher relative to the other, consistent with our data showing that these combinations have impaired replication (FIG. 3B).

### 4. Rep and RepA Impacts on Leaf Cell Death

Plants employ the hypersensitive response as a mechanism to combat viral infection. The hypersensitive response is characterized by a burst of reactive oxygen species and the formation of necrotic lesions resulting from programmed cell death. As viral proteins are often contributors to cell death, the individual contribution of BeYDV proteins to plant leaf necrosis was investigated.

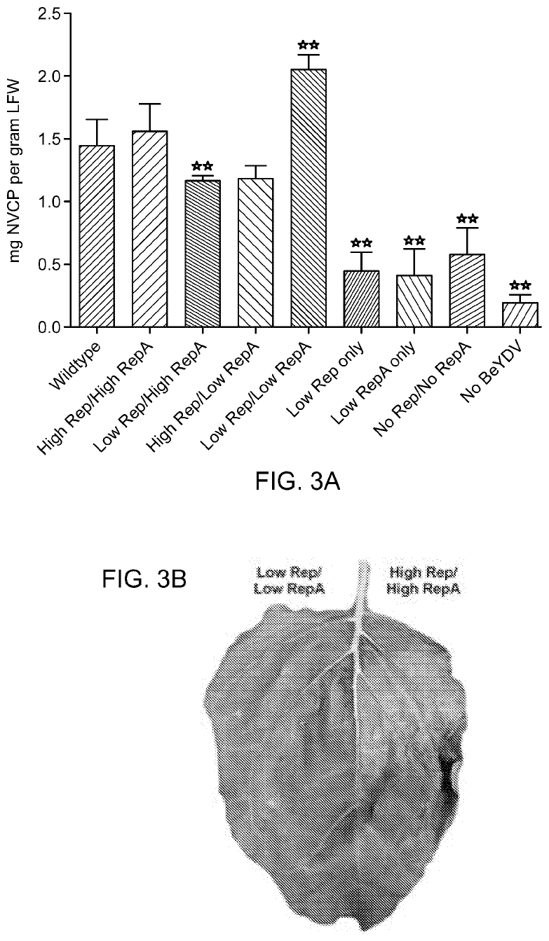

Vectors using the strong 35S promoter to express either Rep, RepA, the movement and coat proteins from BeYDV, or GFP were individually agroinfiltrated into N. benthamiana leaves and monitored for leaf tissue health. Both Rep and RepA produced chlorotic leaf tissue by 3-5 DPI which developed signs of leaf browning and eventually progressed to necrotic lesions by 6-10 DPI, whereas the movement protein, coat protein, and GFP did not produce any notable symptoms (FIG. 4A). The progression of leaf necrosis was greater for Rep than RepA, and the development of necrosis was quicker in older leaves than in younger leaves. BeYDV Rep and RepA both contribute to leaf cell death, while the BeYDV MP and CP did not produce notable symptoms (FIG. 4A). Furthermore, our data is suggestive of vector replication itself as a further contributor to cell death. Viral DNA sensors are well studied components of the innate immune system in animal cells (Takeuchi and Akira, 2009); however, similar sensors have not thus far been identified in plants (Zvereva and Pooggin, 2012).

Many DNA viruses have been shown to activate the DNA damage response during replication (Luftig, 2014). Thus, replicon amplification itself might contribute to leaf necrosis. The vector pRep110, which expresses Rep/RepA together in the native configuration and is insufficient to cause significant cell death on its own, was coinfiltrated with either pBY-EMPTY, which contains the cis-elements necessary for replication but with gene coding sequences replaced with a terminator, or pPS1, which contains no replication elements. Leaf spots infiltrated with pBY-EMPTY and pRep110 produced chlorotic leaf tissue after 3-4 DPI, and necrotic leaf tissue after 6-8 DPI, whereas leaf spots infiltrated with pPS1 and Rep/RepA did not produce necrotic tissue up to 10 DPI (FIG. 4B). Thus, when Rep/RepA are supplied to an empty vector that has had all gene products removed but is still capable of accumulating viral replicons, the cell death response is enhanced compared to when Rep/RepA are supplied to a vector incapable of replicating (FIG. 4B).

### 5. Effects of Reducing Rep/RepA Expression on Expression of Toxic Proteins

To determine whether a modest reduction in Rep/RepA would also benefit the expression of other transgenes, reduced Rep/RepA vectors were coinfiltrated with either pBY-2e-GFP, encoding GFP, or with pBY-2e-MRtx encoding the heavy and light chains of the monoclonal antibody rituximab. These vectors were compared to replicating vectors containing Rep/RepA in the wildtype configuration driven by the native LIR promoter: pBYR2e-GFP and pBYR2e-MRtx. It was previously shown that pBYR2e-GFP accumulates high levels of GFP (Diamos et al., 2016). While GFP is known to be well tolerated even when produced at very high levels in N. benthamiana leaves, the monoclonal antibody rituximab was found to induce a strong cell death response with BeYDV vectors (Diamos et al., 2016). A small but statistically insignificant decrease was observed in GFP expression when low Rep/RepA were supplied, compared to high Rep/RepA or wildtype, and no cell death was observed with any vector (FIG. 5A, and data not shown). By contrast, heavy cell death was observed when rituximab was expressed with wildtype or high Rep/RepA, but not when Rep/RepA were reduced, and this reduction in cell death was correlated with a notable ˜2-fold increase in antibody accumulation (FIG. 5B/C). These results suggest that reducing Rep/RepA from the wildtype level enhances the production of otherwise toxic proteins.

Accordingly, using a controlled reduction in Rep/RepA expression, leaf cell death caused by geminiviral replicons is alleviated (FIGS. 3B and 5C). Despite reducing the number of available DNA templates for transcription, there was minimal reduction in the total yield of recombinant protein with nontoxic proteins (FIG. 5A) and increased accumulation of otherwise toxic proteins (FIGS. 3A, 5B, and 6C). Several hypotheses may explain this observation. BeYDV vectors have replaced the viral movement and coat proteins with an expression cassette containing the gene of interest. During native BeYDV infection, the coat protein results in the accumulation of single-stranded viral DNA, which is packaged into virions, shuttled out of the nucleus, and, in concert with the movement protein, facilitates cell-to-cell movement and systemic spread of viral DNA (Liu et al., 2001). These interactions reduce the amount of double-stranded viral DNA available for transcription. As modified BeYDV expression vectors do not contain the movement and coat proteins, the amount of double-stranded DNA available in the nucleus to serve as a transcription template may exceed wildtype levels. Furthermore, BeYDV vectors also contain the RNA silencing suppressor P19, which likely increases the expression of Rep and RepA relative to wildtype levels. Taken together, these data suggest that more viral replicons are produced than are needed to saturate the plant transcription machinery. Therefore, reducing Rep and/or RepA expression may reduce the plant hypersensitive response while enough DNA templates to drive maximal transcription is still produced. By alleviating the hypersensitive response, further protein accumulation is possible for genes that otherwise would have had their production limited by cell death. Additionally, as RNA silencing and the hypersensitive response are interrelated pathways that act in concert against invading viruses, reducing the onset of hypersensitive response may also prevent premature silencing of BeYDV vectors (Zvereva and Pooggin, 2012).

### 6. Impacts of Point Mutation in Rep/RepA Translation Initiation Site on Replication, Leaf Cell Death, and Transgene Expression

While cell death was reduced and antibody yield was increased by reducing Rep/RepA expression, it required coinfiltration of three separate Agrobacterium vectors. As the native Rep gene also controls the optimum ratio of Rep/RepA by intron splicing, we reasoned that a mutation in the 5′ UTR of Rep/RepA would be a simple modification to simultaneously reduce expression of both genes while maintaining the native mechanism of controlling the relative production of Rep/RepA. The sequence context around the initiation site plays a critical role in translation (Kozak, 1999). Experiments with tobacco cells found that altering the initiation context from CAUAUGC to AAUAUGG (start codon underlined) resulted in a 4-fold increase in gene expression (Ayre, 2002).

To construct a simplified vector with reduced expression of Rep and RepA, single nucleotide mutations were created in the native 5′ UTR of Rep/RepA at the −3 position from the Rep/RepA start codon. These mutations were designed to provide a less favorable sequence context for translation initiation, which has been shown to favor A or G in the −3 position for dicot plants (Sugio et al., 2010). The resulting vector contains an AAUAUG to CAUAUG mutation.

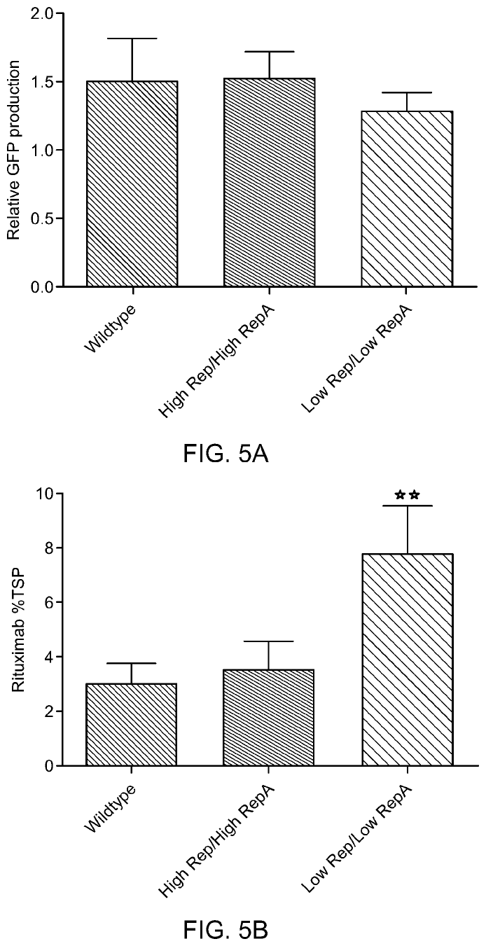

An AACATG to CACATG mutation (where ATG indicates the rep start codon) reduced both Rep/RepA accumulation (FIG. 6A) and replicon amplification (FIG. 6B) by approximately 40%, similar to the results observed with low-expressing separated Rep/RepA vectors. To characterize expression and cell death with this vector, rituximab was produced with or without the mutation. As expected, the Rep/RepA mutant had reduced cell death (FIG. 6C) and increased antibody production, reaching 10% TSP or approximately 0.8-1.0 g rituximab per kg leaf tissue (FIG. 6C). Accordingly, the vector containing above described point mutation in the translation initiation reduced Rep/RepA expression, reduced cell death, and provided enhanced expression of toxic proteins.

These results also indicate that vector replication can be reduced with a single change from the wildtype Rep/RepA gene. As multiple BeYDV replicons can be placed in tandem on the same T-DNA (Huang et al., 2010), this strategy can be used to produce heteromultimeric proteins from a single vector.

### 7. Comparison of Agrobacterium Concentrations Needed for Replicating Vectors and Nonreplicating Vectors

Agrobacterium contributes to the plant cell death response in a complex manner (Hwang et al., 2015), though infiltration with higher Agrobacterium concentrations has often been found to contribute to cell death (Wroblewski et al., 2005). While an Agrobacterium OD₆₀₀ of ˜0.2 is sufficient to deliver T-DNA to the majority of plant cells, nonreplicating vector systems often use much higher concentrations of Agrobacterium to achieve optimum expression. This may be due to the delivery of multiple DNA copies to each cell, which serve as additional transcription templates. As replicating systems greatly amplify the input T-DNA, additional copies would be unnecessary. In N. benthamiana leaves, Agrobacterium strain EHA105 reduces leaf necrosis relative to other commonly used Agrobacterium strains when used to deliver replicating BeYDV vectors (Diamos et al. 2016). Many nonreplicating vector systems use high Agrobacterium concentrations of around an OD₆₀₀ of 1.2 (Sainsbury et al., 2009).

To investigate the relationship between Agrobacterium concentration and vector replication, a replicating BeYDV vector expressing GFP was infiltrated at various Agrobacterium concentrations. No significant differences in GFP expression were observed until the OD₆₀₀ was reduced below 0.2 (FIG. 7A). By contrast, GFP expression with pEAQ-HT-GFP (Sainsbury et al., 2009) was reduced by nearly half when the Agrobacterium OD₆₀₀ was decreased from 1.2 to 0.2 (FIG. 7B). This observation agrees with the observation in Sainsbury et al. (2009). By contrast, we found no reduction in yield by reducing the Agrobacterium concentration from 1.2 to 0.2 using replicating BeYDV vectors (FIG. 7A/7B). While GFP was well tolerated at all Agrobacterium concentrations tested, the added Agrobacterium load may be less tolerable with more toxic proteins.

To further evaluate the relationship between Agrobacterium concentration and cell death, replicating BeYDV vectors expressing hepatitis B core antigen tandem-linked heterodimers (Peyret et al., 2015) were infiltrated at decreasing Agrobacterium concentrations. Agrobacterium OD₆₀₀ concentrations of 1.6 and 0.8 produced visible leaf necrosis, while 0.4 and 0.2 did not (FIG. 7C). Taken together, these data show that replicating BeYDV vectors provide optimal expression with lower Agrobacterium concentrations than nonreplicating vectors, allowing further reductions in cell death.

For the expression of toxic proteins, inventors observed that necrosis developed when using higher Agrobacterium concentrations, but not with lower concentrations (FIG. 7D). That this relationship was observed only with certain proteins suggests that cell death only occurs when the combined action of multiple necrosis-inducing factors reach a specific threshold.

### 8. Viral Flanking Regions Impact on Leaf Cell Death

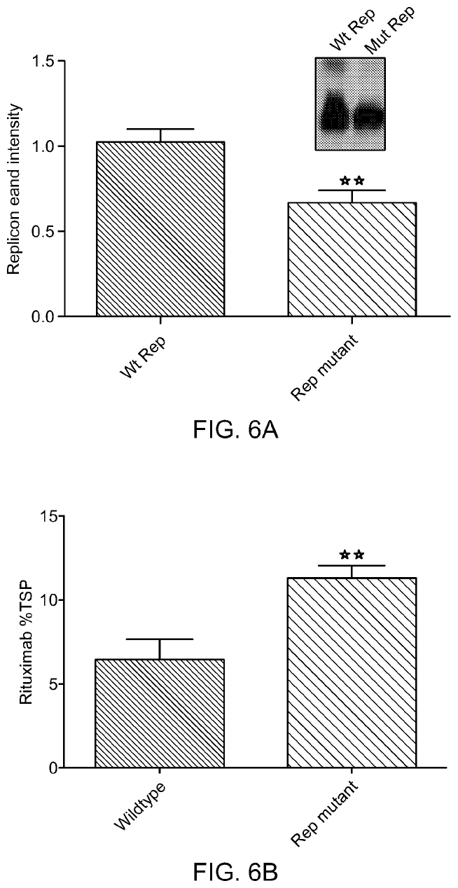

While no substantial necrosis developed with either BeYDV or pEAQ vectors expressing GFP, leaf chlorosis appeared only with pEAQ-HT-GFP, an effect which was more pronounced at higher Agrobacterium concentrations (FIG. 8A). pEAQ vectors contain the 5′ and 3′ UTRs from cowpea mosaic virus, so other viral UTRs may contribute to cell death. The 5′ UTR from tobacco mosaic virus was found to increase the cell death response compared to the native N. benthamiana NbPsaK 5′ UTR, despite the TMV 5′ UTR producing less recombinant protein (FIG. 8B and Diamos et al. 2016). The 5′ and 3′ UTRs from pea enation mosaic virus also substantially increased cell death, while those from barley yellow dwarf virus did not (FIG. 8C). These data show that certain viral untranslated regions increase the cell death response in N. benthamiana leaves. In particular, viral UTRs contribute substantially to cell death, while a native plant-derived 5′ UTR does not.

### 9. Comparison of Mutations in the 5′ UTR of Rep/RepA on Recombinant Protein Expression

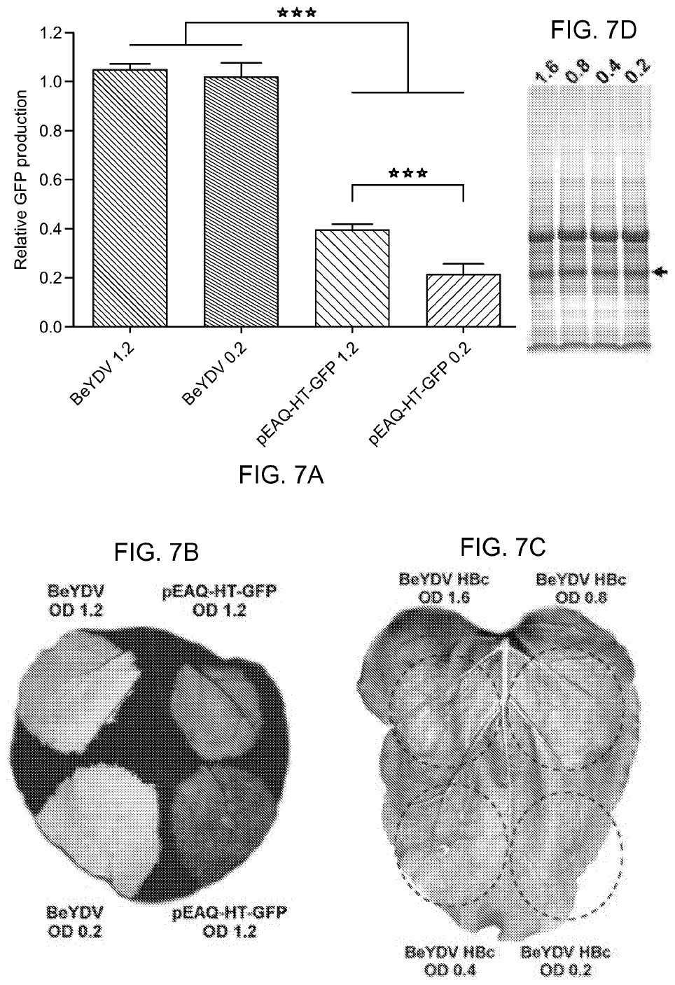

Constructs containing mutations in the 5′ UTR of Rep/RepA with the goal of reducing expression of a recombinant protein in plants. pBYe-R1-GFP (R1 in FIG. 9) has a mutation at −1 (relative to ATG start codon) of the Rep/RepA 5′ UTR (AACATG to AAAATG). pBYe-R2-GFP (R2 in FIG. 9) has a mutation at −3 (AACATG to CACATG). pBYe-R3-GFP (R3 in FIG. 9) has a different mutation at −3 mutation (AACATG to TACATG). To show the effects of mutants on the abundance of Rep protein, soluble proteins were extracted and fractionated by SDS-PAGE, and GFP expression was detected by western blot with anti-Rep rabbit serum. As shown in FIG. 9, R1 had no discernable effect, while R2 and R3 reduced Rep protein substantially.

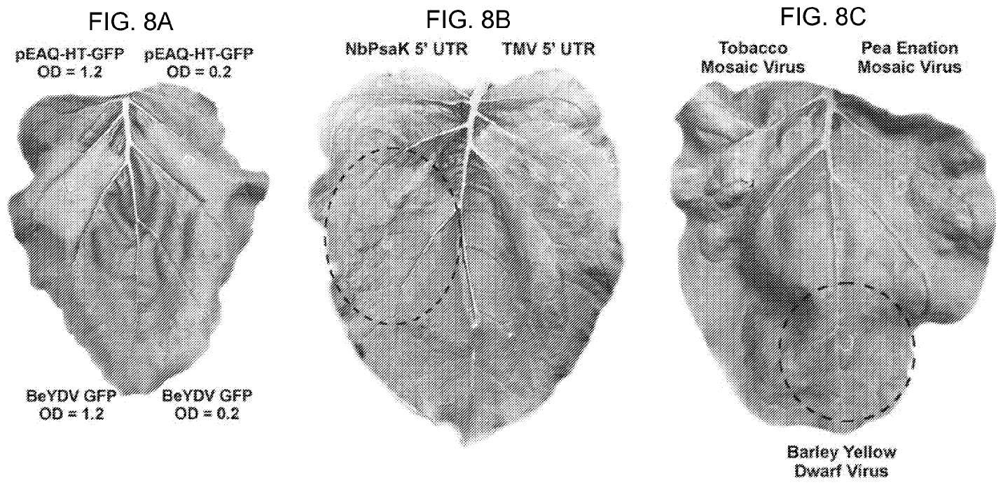

To evaluate the effects of the mutants on replicon DNA abundance, DNA was extracted from the plants expression and quantified using and performed agarose gel quantification. FIG. 10 shows that A→C mutation (R2) or A→T mutation (R3) at the −3 position of Rep/RepA reduced replication to the same extent, while the C→A mutation at the −1 position (R1) had very little effect.

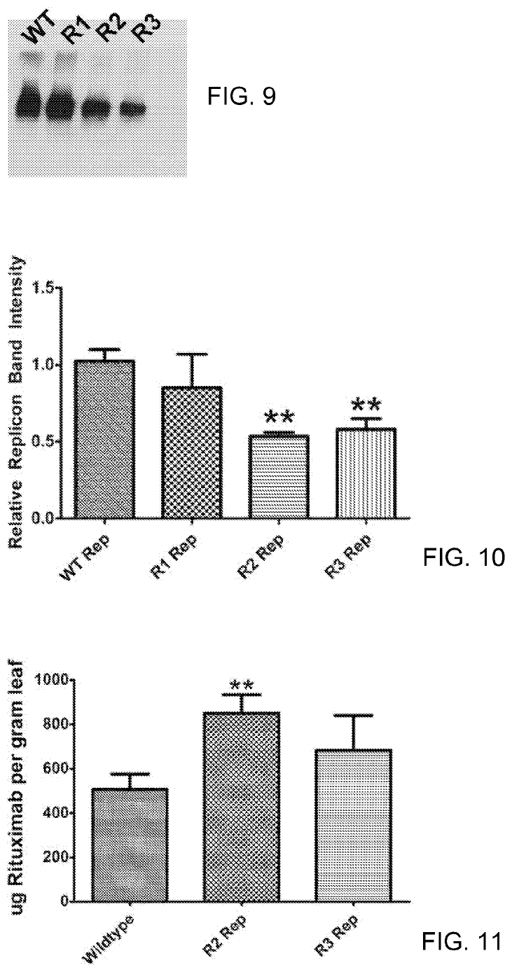

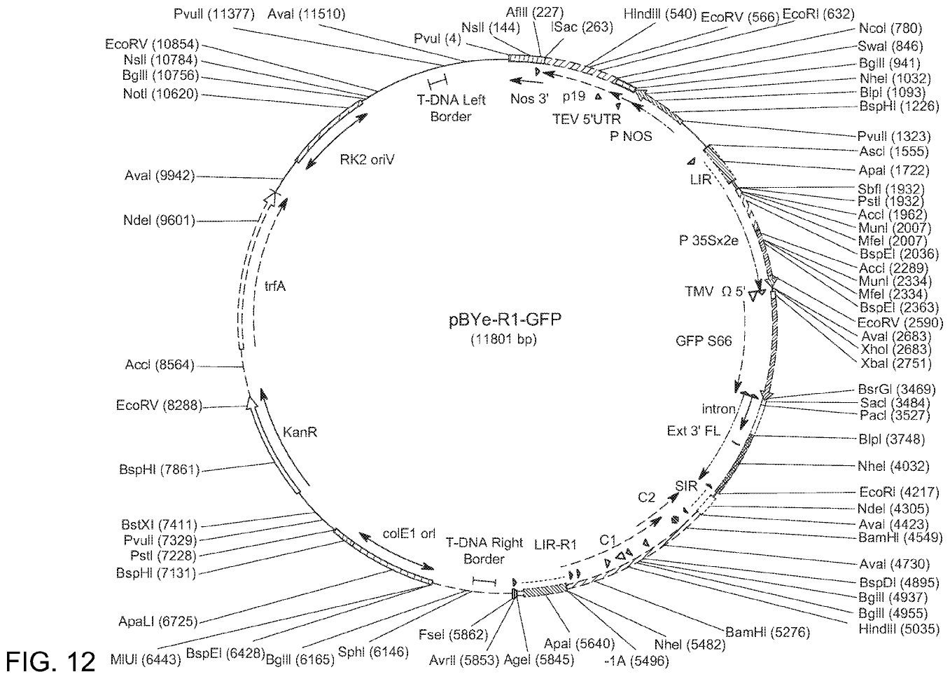

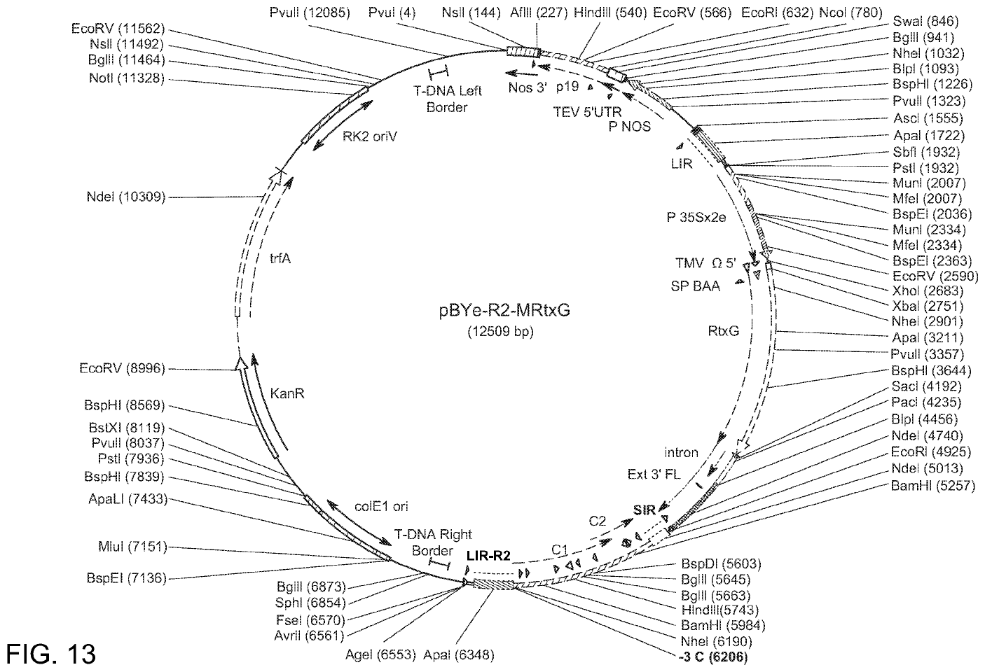

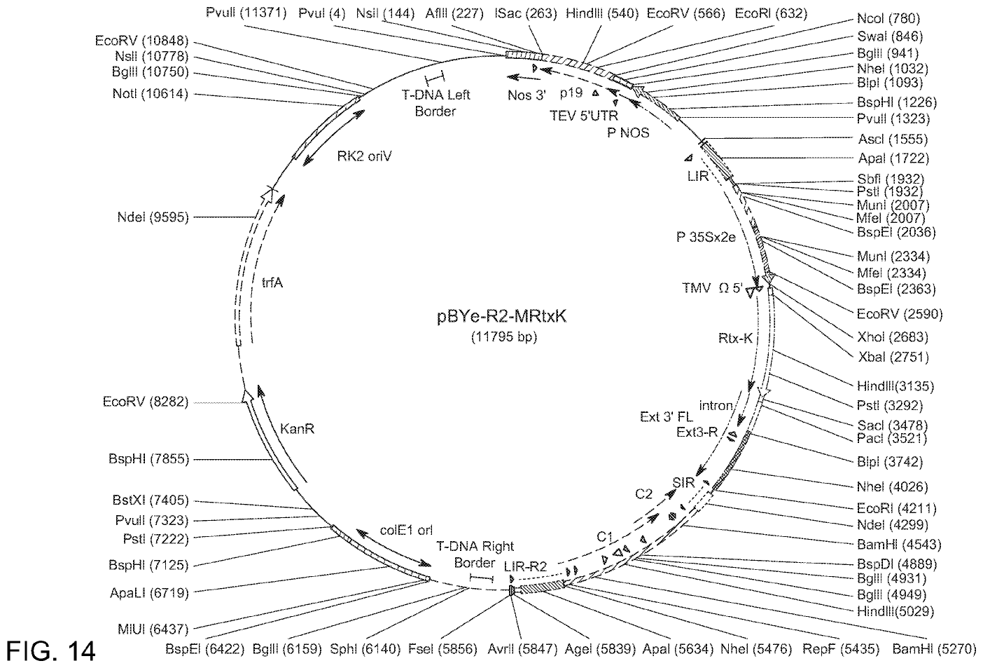

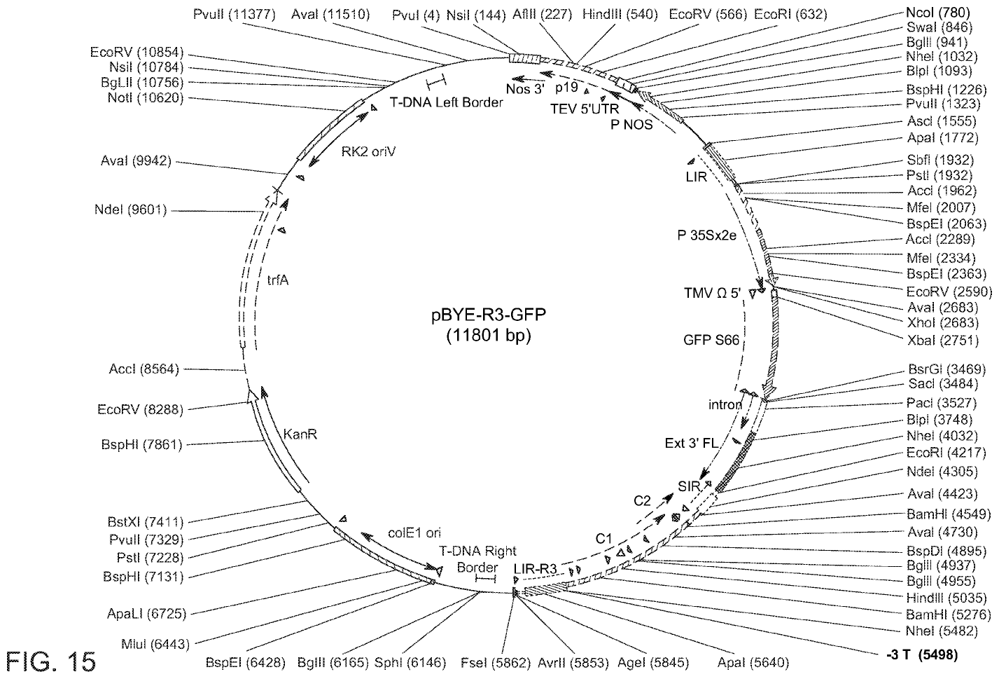

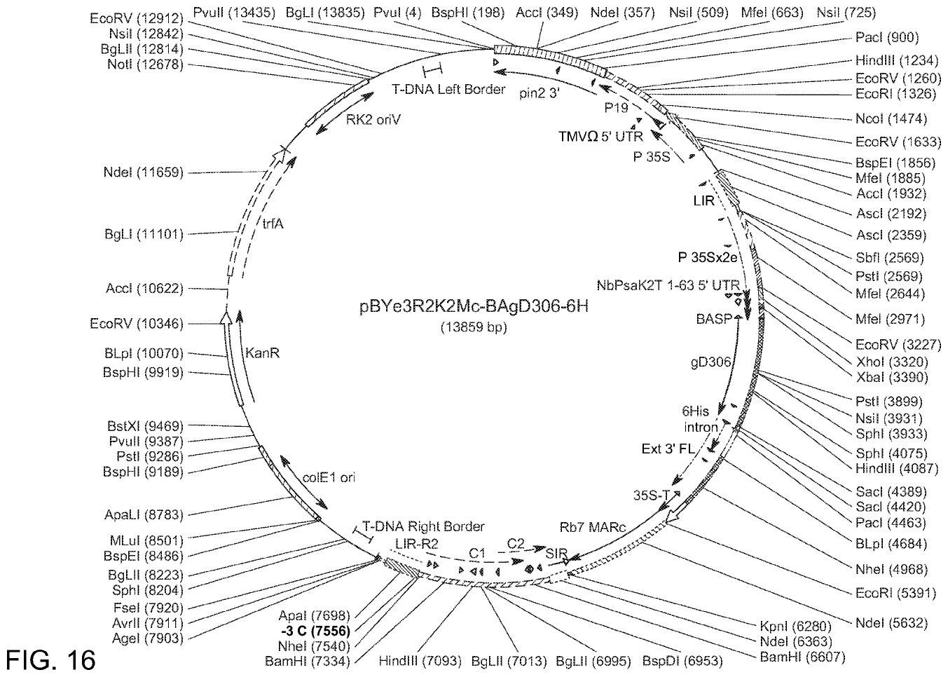

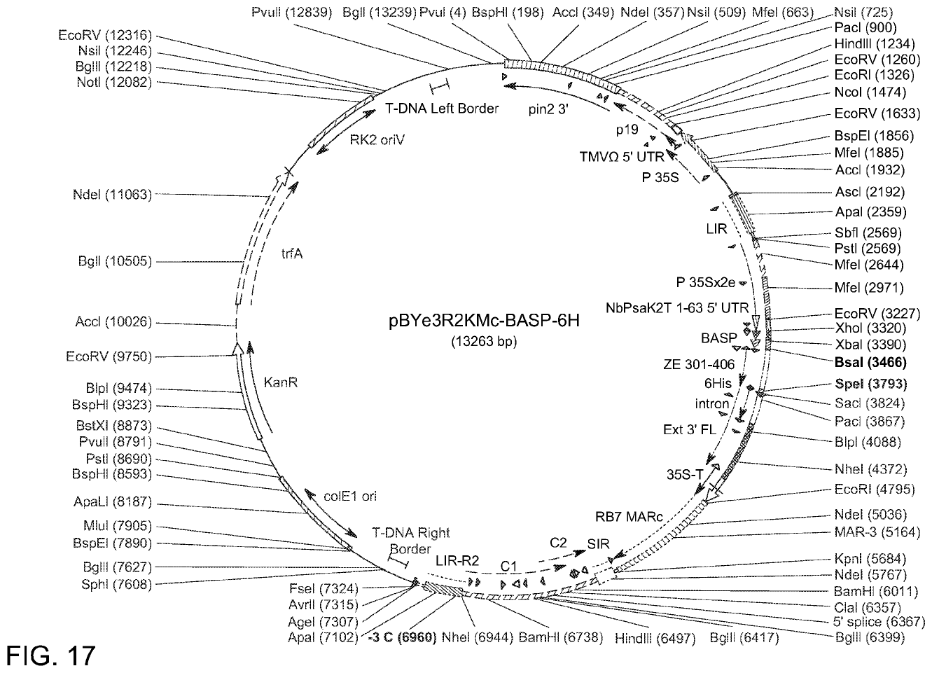

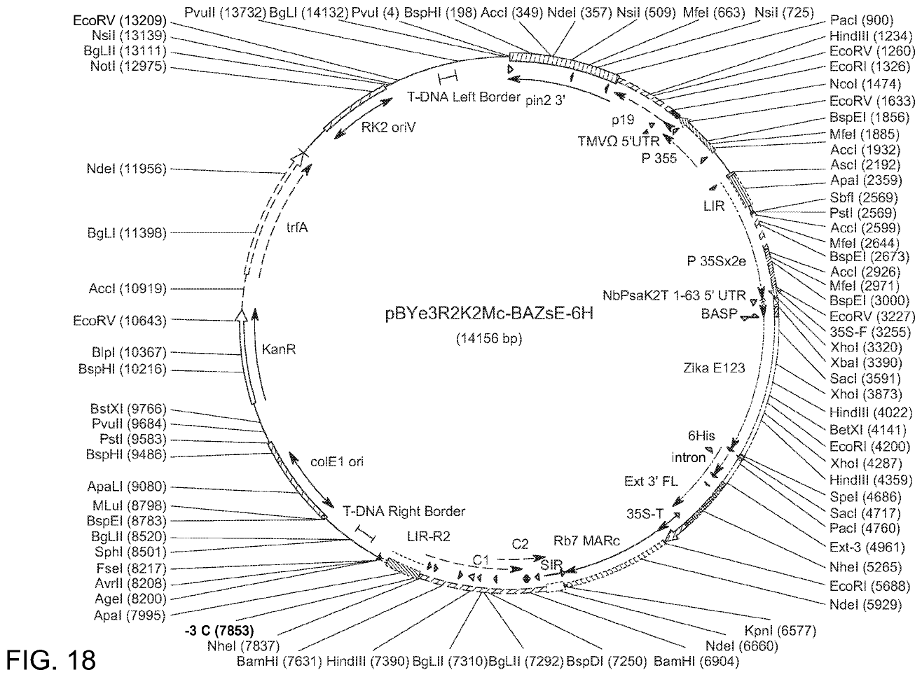

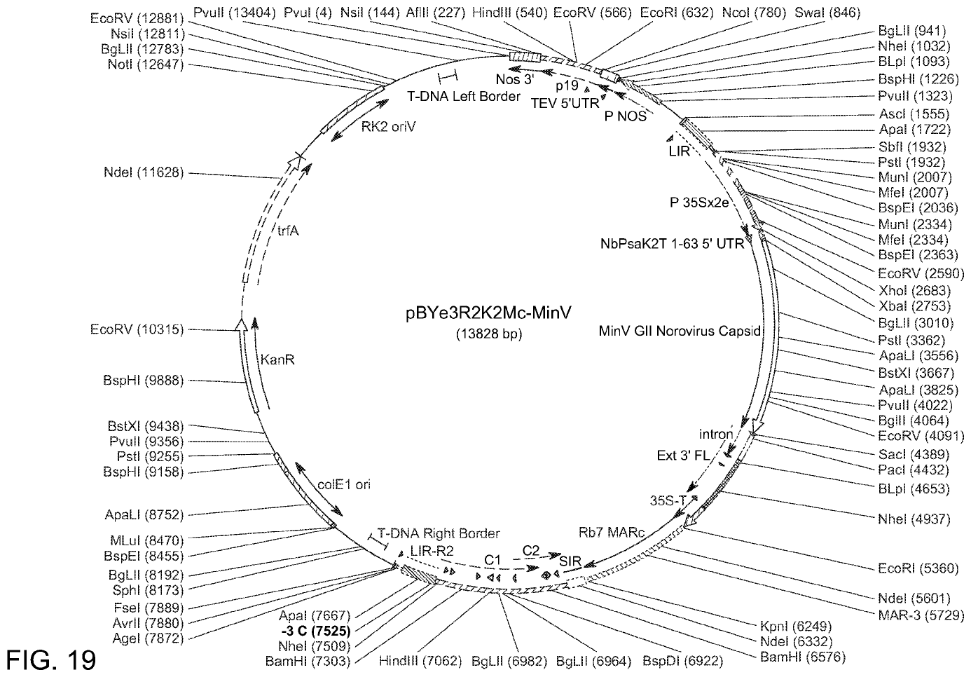

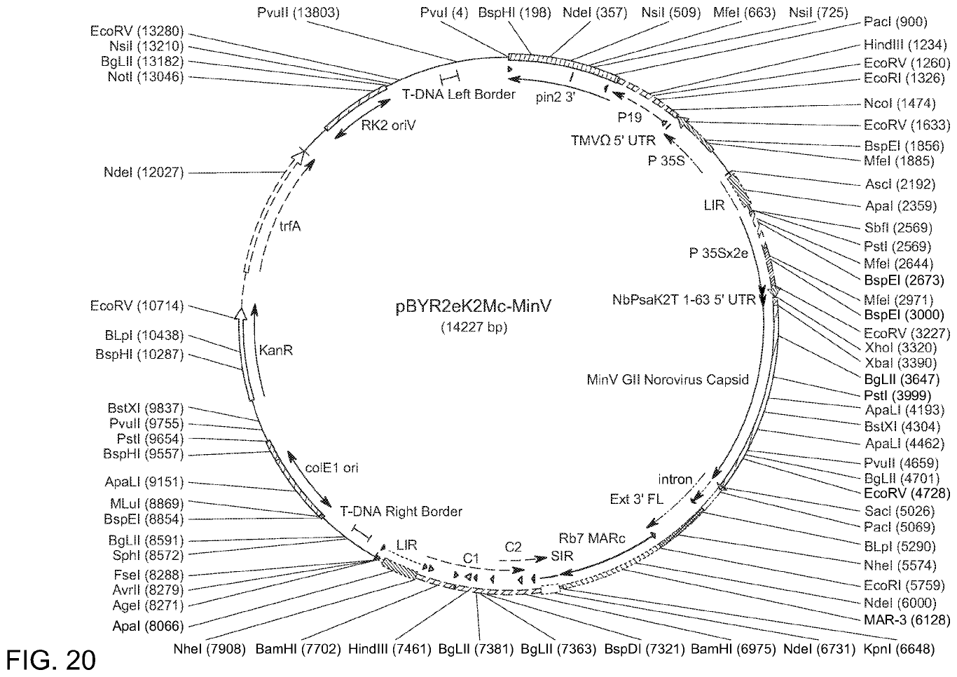

Rep mutant vectors for expression of rituximab heavy and light chains were constructed in order to evaluate effects of mutations in the 5′ UTR of Rep/RepA on rituximab expression and cell death. pBYe-R2-MRtxG and pBYe-R2-MrtxK contain a mutation at −3 (relative to ATG start codon) of the Rep/RepA 5′ UTR (AACATG to CACATG; R2 Rep in FIG. 11), while pBYe-R3-MRtxG and pBYe-R3-MRtxK contain a mutation at −3 (AACATG to TACATG; R3 Rep in FIG. 11). Both R2 Rep and R3 Rep performed better than wild-type, but only R2 Rep was statistically significant with this sample size (FIG. 11). Both R2 Rep and R3 Rep had less cell death than the wildtype vector. These results show that a modest Rep/RepA reduction enhances transgene production, which may be due to a reduction of cell death resulting from excess replicons and the toxic effects of Rep/RepA.

### 10. Materials and Methods

a. Vector Construction

A series of expression vectors containing promoters of varying strengths were created to express Rep and RepA. The Ubi3 promoter was obtained from pUbi3-GUS (Garbarino and Belknap, 1994) by BseRI (T4 blunt) PstI digestion, and ligated into pRep110 (Huang et al., 2009) digested SbfI (T4 blunt) and XhoI, to create pRep107. The Ubi3 promoter with ubiquitin fusion was excised from pUbi3-GUS by PstI-NcoI digestion and ligated into pRep110 digested SbfI-SacI along with C1/C2 excised from pBY036 digested NcoI-SacI to create pRep106. The soybean vspB promoter was obtained from pGUS220 (Mason et al., 1993) by HindIII-NcoI digestion and ligated with pRep110 digested HindIII-SacI and pBY034 digested NcoI-SacI to create pRep108. The Agrobacterium nopaline synthase (NOS) promoter was obtained from pGPTV-Kan (Becker et al., 1992) by HindIII-NcoI digestion and ligated into pBI101 (Jefferson et al., 1987) along with C1/C2 excised from pBY036 digested NcoI-SacI to create pRep111.

The intron-deleted form of BeYDV rep was previously described (Mor et al., 2003). For RepA vectors, the sequence following the RepA stop codon was deleted and an additional stop codon was inserted in the Rep reading frame to prevent further translation. To accomplish this, a primer RepA-Sac-R (5′-CGGAGCTCTATGTTAATTGCTTCCACAATGGGAC; SEQ ID NO. 1) designed to insert a stop codon and create a SacI site at the end of the RepA coding sequence was used to amplify RepA from pRep110 along with primer TEV (5′-GCATTCTACTTCTATTGCAGC; SEQ ID NO. 2). The product was digested ClaI-SacI and ligated into pRep110 digested likewise to yield pRepA110. XhoI-SacI or NcoI-SacI fragments containing either the deleted intron form of Rep excised from pBY037, or RepA excised from pRepA110, were ligated into expression vectors containing the promoters Ubi (pRep106), UbiF (pRep107), VspB (pRep108), or NOS (pRep111) to generate Rep and RepA expressing vectors.

To create BeYDV expression vectors that required Rep/RepA to be supplied in trans, Rep/RepA were deleted from the Norwalk virus capsid protein (NVCP)-expressing vector pBYR2e-sNV or the rituximab-expressing vector pBYR2e-MRtx (Diamos et al., 2016) by BamHI digestion and self-ligation of the backbone vector to yield pBY-2e-sNV and, pBY-2e-MRtx respectively. The empty replicon vector pBY-EMPTY was created by excising the PstI-SacI fragment from pKS-RT38, which contains the potato pinII terminator region derived from pRT38 (Thornburg et al., 1987), and ligating it into pBY-GFP (Huang et al., 2009) digested SbfI-SacI. To introduce a AACATG to CACATG mutation to the 5′ UTR of Rep/RepA, the primer LIRc-Nhe2-R (5′-taGCTAGCAGAAGGCATGTGGTTGTGACTCCGAGGGGTTG; SEQ ID NO. 3) containing the mutation was used to amplify the modified LIR from pBY027 with primer M13F. The PCR product was digested NheI-AgeI and ligated into pBYR2e-GFP digested BspDI-AgeI along with the rep-containing NheI-BspDI fragment from pBYR2e-GFP to create pBY-R2-GFP. Vectors containing NbPsaK, PEMV and BYDV 3′ and 5′ UTRs were previously described (Diamos et al., 2016; Diamos and Mason, 2018).

b. Agroinfiltration of N. benthamiana Leaves

Binary vectors were separately introduced into Agrobacterium tumefaciens GV3101 or EHA105 by electroporation. The resulting strains were verified by restriction digestion or PCR, grown overnight at 30° C., and used to infiltrate leaves of 5- to 6-week-old N. benthamiana maintained at 23-25° C. Briefly, the bacteria were pelleted by centrifugation for 5 min at 5,000 g and then resuspended in infiltration buffer (10 mM 2-(N-morpholino)ethanesulfonic acid (IVIES), pH 5.5 and 10 mM MgSO4) to OD₆₀₀=0.2, unless otherwise described. When mixing two constructs, each Agrobacterium concentration was instead set to OD₆₀₀=0.4, and then mixed 1:1. Similarly, for three constructs, each was set to OD₆₀₀=0.6, and mixed 1:1:1. The resulting bacterial suspensions were injected by using a syringe without needle into fully expanded leaves (9-12 cm long) through a small puncture (Huang et al. 2004). Plant tissue was harvested after 5 DPI, or as stated for each experiment. Leaves producing GFP were photographed under UV illumination generated by a B-100AP lamp (UVP, Upland, Calif., USA).

c. Protein Extraction

Total protein extract was obtained by homogenizing agroinfiltrated leaf samples with 1:5 (w:v) ice cold extraction buffer (25 mM sodium phosphate, pH 7.4, 100 mM NaCl, 1 mM EDTA, 0.1% Triton X-100, 10 mg/mL sodium ascorbate, 0.3 mg/mL phenylmethylsulfonyl fluoride) using a Bullet Blender machine (Next Advance, Averill Park, N.Y., USA) following the manufacturer's instruction. To enhance solubility, homogenized tissue was rotated at room temperature or 4° C. for 30 minutes. The crude plant extract was clarified by centrifugation at 13,000 g for 10 min at 4° C. Necrotic leaf tissue has reduced water weight, which can lead to inaccurate measurements based on leaf mass. Therefore, extracts were normalized based on total protein content by Bradford protein assay kit (Bio-Rad, Hercules, Calif., USA) with bovine serum albumin as standard.

d. SDS-PAGE and Western Blot

Clarified plant protein extract was mixed with sample buffer (50 mM Tris-HCl, pH 6.8, 2% SDS, 10% glycerol, 0.02% bromophenol blue) and separated on 4-15% polyacrylamide gels (Bio-Rad, Hercules, Calif., USA). For reducing conditions, 0.5 M dithiothreitol was added, and the samples were boiled for 10 min prior to loading. Polyacrylamide gels were either transferred to a PVDF membrane or stained with Coomassie stain (Bio-Rad, Hercules, Calif., USA) following the manufacturer's instructions. For Rep/RepA detection, the protein transferred membranes were blocked with 5% dry milk in PBST (PBS with 0.05% tween-20) for 1 h at 37° C. and probed in succession with rabbit anti-Rep (antibodies raised against an N-terminal 154 amino acid fragment of Rep/RepA) diluted 1:2000 and goat anti-rabbit IgG-horseradish peroxidase conjugated (Sigma-Aldrich, St. Louis, Mo., USA) diluted 1:10,000 in 1% PBSTM. Bound antibody was detected with ECL reagent (Amersham, Little Chalfont, United Kingdom). For GFP detection, the 26 kDa fluorescent GFP band was quantified by gel densitometry using ImageJ software.

e. Protein Quantification by ELISA

GI and GII norovirus capsid concentration was analyzed by sandwich ELISA. A rabbit polyclonal anti-GI or anti-GII antibody was bound to 96-well high-binding polystyrene plates (Corning, Corning, N.Y., USA), and the plates were blocked with 5% nonfat dry milk in PBST. After washing the wells with PBST (PBS with 0.05% Tween 20), the plant extracts were added and incubated. The bound norovirus capsids were detected by incubation with guinea pig polyclonal anti-GI or anti-GII antibody followed by goat anti-guinea pig IgG-horseradish peroxidase conjugate. The plate was developed with TMB substrate (Thermo Fisher Scientific, Waltham, Mass., USA) and the absorbance was read at 450 nm. Plant-produced GI or GII capsids were used as the reference standard (Kentucky Bio Processing, Kentucky, USA).

For rituximab quantification, plant protein extracts were analyzed by ELISA designed to detect the assembled form of mAb (with both light and heavy chains) as described previously (Giritch et al. 2006). Briefly, plates were coated with a goat anti-human IgG specific to gamma heavy chain (Southern Biotech, Birmingham, Ala., USA). After incubation with plant protein extract, the plate was blocked with 5% non-fat dry milk in PBST, then incubated with a HRP-conjugated anti-human-kappa chain.

f. Plant DNA Extraction and Replicon Quantification

Total DNA was extracted from 0.1 g plant leaf samples using the DNeasy Plant Mini Kit (Qiagen) according to the manufacturer's instructions. DNA (˜1 μg) was separated on 1% agarose gels stained with ethidium bromide. The replicon DNA band intensity was quantified using ImageJ software, using the high molecular weight plant chromosomal DNA band as an internal loading control. Columns represent means±standard deviation from 3 or more independently infiltrated samples.

g. RT-PCR

Total RNA was extracted from 0.1 g leaf samples using the RNeasy Plant Mini Kit (Qiagen) according to the manufacturer's instructions. Residual DNA was removed using the DNA-Free system (Ambion). First-strand cDNA was synthesized from 1 μg of total RNA primer using the Superscript III First Strand Synthesis System (Invitrogen) according to the manufacturer's instructions using oligo dT22 primer. RT-PCR was performed using primers RepF (5′-ACCCCAAGTGCTCATCTC) and RepR1 (5′-GCGACACGTACTGCTCA) to detect Rep and RepA transcripts.

### REFERENCES CITED

Almon, E., Horowitz, M., Wang, H. L., Lucas, W. J., Zamski, E., and Wolf, S. (1997). Phloem-Specific Expression of the Tobacco Mosaic Virus Movement Protein Alters Carbon Metabolism and Partitioning in Transgenic Potato Plants. Plant Physiol. 115, 1599-1607. doi:115/4/1599 [pii].
Ayre, B. G. (2002). Optimization of trans-splicing ribozyme efficiency and specificity by in vivo genetic selection. Nucleic Acids Res. 30, 141e-141. doi:10.1093/nar/gnf141.
Becker, D., Kemper, E., Schell, J., and Masterson, R. (1992). New plant binary vectors with selectable markers located proximal to the left T-DNA border. Plant Mol. Biol. 20, 1195-1197. doi:10.1007/BF00028908.
Boller, T., and Felix, G. (2009). A Renaissance of Elicitors: Perception of Microbe-Associated Molecular Patterns and Danger Signals by Pattern-Recognition Receptors. Annu. Rev. Plant Biol. 60, 379-406. doi:10.1146/annurev.arplant.57.032905.105346.
Bonardi, V., Cherkis, K., Nishimura, M. T., and Dangl, J. L. (2012). A new eye on NLR proteins: focused on clarity or diffused by complexity? Curr. Opin. Immunol. 24, 41-50. doi:10.1016/j.coi.2011.12.006.
Brendolise, C., Montefiori, M., Dinis, R., Peeters, N., Storey, R. D., and Rikkerink, E. H. (2017). A novel hairpin library-based approach to identify NBS-LRR genes required for effector-triggered hypersensitive response in Nicotiana benthamiana. Plant Methods 13, 32. doi:10.1186/s13007-017-0181-7.
Chen, Q., and Davis, K. R. (2016). The potential of plants as a system for the development and production of human biologics. F1000Research 5, 912. doi:10.12688/f1000research.8010.1.
Choudhury, N. R., Malik, P. S., Singh, D. K., Islam, M. N., Kaliappan, K., and Mukherjee, S. K. (2006). The oligomeric Rep protein of Mungbean yellow mosaic India virus (MYMIV) is a likely replicative helicase. Nucleic Acids Res. 34, 6362-6377. doi:10.1093/nar/gk1903.
Clerot, D., and Bernardi, F. (2006). DNA Helicase Activity Is Associated with the Replication Initiator Protein Rep of Tomato Yellow Leaf Curl Geminivirus. J. Virol. 80, 11322-11330. doi:10.1128/JVI.00924-06.
Collin, S., Fernandez-Lobato, M., Gooding, P. S., Mullineaux, P. M., and Fenoll, C. (1996). The two nonstructural proteins from wheat dwarf virus involved in viral gene expression and replication are retinoblastoma-binding proteins. Virology 219, 324-9. doi:10.1006/viro.1996.0256.
Dangl, J. L., and Jones, J. D. G. (2001). Plant pathogens and integrated defence responses to infection. Nature 411, 826-833. doi:10.1038/35081161.
Dawson, W. O. (1999). Tobacco mosaic virus virulence and avirulence. Philos. Trans. R. Soc. B Biol. Sci. 354, 645-651. doi:10.1098/rstb.1999.0416.
Diamos, A. G. A. G., and Mason, H. S. H. S. (2018). Chimeric 3′ Flanking Regions Strongly Enhance Gene Expression in Plants. Plant Biotechnol. J. doi:10.1111/pbi.12931.
Diamos, A. G. A. G., Rosenthal, S. H. S. H., and Mason, H. S. H. S. (2016). 5′ and 3′ Untranslated Regions Strongly Enhance Performance of Geminiviral Replicons in Nicotiana benthamiana Leaves. Front. Plant Sci. 7, 200. doi:10.3389/fpls.2016.00200.
Dodds, P. N., and Rathjen, J. P. (2010). Plant immunity: towards an integrated view of plant-pathogen interactions. Nat. Rev. Genet. 11, 539-548. doi:10.1038/nrg2812.
Garbarino, J. E., and Belknap, W. R. (1994). Isolation of a ubiquitin-ribosomal protein gene (ubi3) from potato and expression of its promoter in transgenic plants. Plant Mol. Biol. 24, 119-127. doi:10.1007/BF00040579.
Garrido-Ramirez, E. R., Sudarshana, M. R., Lucas, W. J., and Gilbertson, R. L. (2000). Bean dwarf mosaic virus BV1 protein is a determinant of the hypersensitive response and avirulence in Phaseolus vulgaris. Mol. Plant. Microbe. Interact. 13, 1184-94. doi:10.1094/MPMI.2000.13.11.1184.
Gleba, Y., Klimyuk, V., and Marillonnet, S. (2005). Magnifection—a new platform for expressing recombinant vaccines in plants. Vaccine 23, 2042-8. doi:10.1016/j.vaccine.2005.01.006.
Gleba, Y. Y., Tuse, D., and Giritch, A. (2014). Plant viral vectors for delivery by Agrobacterium. Curr. Top. Microbiol. Immunol. 375, 155-92. doi:10.1007/82_2013_352.
Gutierrez, C. (1999). Geminivirus DNA replication. Cell. Mol. Life Sci. 56, 313-329. doi:10.1007/s000180050433.
Gutierrez, C., Ramirez-Parra, E., Mar Castellano, M., Sanz-Burgos, A. P., Luque, A., and Missich, R. (2004). Geminivirus DNA replication and cell cycle interactions. Vet. Microbiol. 98, 111-119. doi:10.1016/j.vetmic.2003.10.012.
Hamorsky, K. T., Kouokam, J. C., Jurkiewicz, J. M., Nelson, B., Moore, L. J., Husk, A. S., et al. (2015). N-Glycosylation of cholera toxin B subunit in Nicotiana benthamiana: impacts on host stress response, production yield and vaccine potential. Sci. Rep. 5, 8003. doi:10.1038/srep08003.
Hefferon, K. L. (2003). Independent expression of Rep and RepA and their roles in regulating bean yellow dwarf virus replication. J. Gen. Virol. 84, 3465-3472. doi:10.1099/vir.0.19494-0.
Hiatt, A., Zeitlin, L., and Whaley, K. J. (2014). Plant-Derived Monoclonal Antibodies for Prevention and Treatment of Infectious Disease. Microbiol. Spectr. 2. doi:10.1128/microbiolspec.AID-0004-2012.
Horvath, G. V., Pettko-Szandtner, A., Nikovics, K., Bilgin, M., Boulton, M., Davies, J. W., et al. (1998). Prediction of functional regions of the maize streak virus replication-associated proteins by protein-protein interaction analysis. Plant Mol. Biol. 38, 699-712. doi:10.1023/A:1006076316887.
Howell, S. H. (2013). Endoplasmic Reticulum Stress Responses in Plants. Annu. Rev. Plant Biol. 64, 477-499. doi:10.1146/annurev-arplant-050312-120053.
Huang, Z., Chen, Q., Hjelm, B., Arntzen, C., and Mason, H. (2009). A DNA replicon system for rapid high-level production of virus-like particles in plants. Biotechnol. Bioeng. 103, 706-714. doi:10.1002/bit.22299.
Huang, Z., Phoolcharoen, W., Lai, H., Piensook, K., Cardineau, G., Zeitlin, L., et al. (2010). High-level rapid production of full-size monoclonal antibodies in plants by a single-vector DNA replicon system. Biotechnol. Bioeng. 106, n/a-n/a. doi:10.1002/bit.22652.
Huang, Z., Santi, L., LePore, K., Kilbourne, J., Arntzen, C. J., and Mason, H. S. (2006). Rapid, high-level production of hepatitis B core antigen in plant leaf and its immunogenicity in mice. Vaccine 24, 2506-2513. doi:10.1016/j.vaccine.2005.12.024.
Hwang, E. E., Wang, M. B., Bravo, J. E., and Banta, L. M. (2015). Unmasking host and microbial strategies in the Agrobacterium-plant defense tango. Front. Plant Sci. 6, 200. doi:10.3389/fpls.2015.00200.
Jefferson, R. A., Kavanagh, T. A., and Bevan, M. W. (1987). GUS fusions: beta-glucuronidase as a sensitive and versatile gene fusion marker in higher plants. EMBO J. 6, 3901-7. doi:10.1073/pnas.1411926112.
Jin, M., Li, C., Shi, Y., Ryabov, E., Huang, J., Wu, Z., et al. (2008). A single amino acid change in a geminiviral Rep protein differentiates between triggering a plant defence response and initiating viral DNA replication. J. Gen. Virol. 89, 2636-41. doi:10.1099/vir.0.2008/001966-0.
Jones, J. D. G., and Dangl, J. L. (2006). The plant immune system. Nature 444, 323-329. doi:10.1038/nature05286.
Kim, M.-Y., Reljic, R., Kilbourne, J., Ceballos-Olvera, I., Yang, M.-S., Reyes-del Valle, J., et al. (2015). Novel vaccination approach for dengue infection based on recombinant immune complex universal platform. Vaccine 33, 1830-1838. doi: 10.1016/j.vaccine.2015.02.036.
Kozak, M. (1999). Initiation of translation in prokaryotes and eukaryotes. Gene 234, 187-208. doi:10.1016/S0378-1119(99)00210-3.
Krenz, B., Neugart, F., Kleinow, T., and Jeske, H. (2011). Self-interaction of Abutilon mosaic virus replication initiator protein (Rep) in plant cell nuclei. Virus Res. 161, 194-197. doi: 10.1016/j.virusres.2011.07.020.
Lai, H., He, J., Engle, M., Diamond, M. S., and Chen, Q. (2012). Robust production of virus-like particles and monoclonal antibodies with geminiviral replicon vectors in lettuce. Plant Biotechnol. J.
Lai, H., He, J., Engle, M., Diamond, M. S., and Chen, Q. (2012). No Title. 10, 95-104. doi: 10.1111/j 0.1467-7652.2011.00649.x.
Liu, H., Andrew, Lucy, P., Davies, J. W., Boulton, M. I., Lucy, A. P., et al. (2001). A single amino acid change in the coat protein of Maize streak virus abolishes systemic infection, but not interaction with viral DNA or movement protein. Mol. Plant Pathol. 2, 223-228. doi:10.1046/j.1464-6722.2001.00068.x.
Liu, L., Davies, J. W., Stanley, J., and Davies, J. W. (1998). Mutational analysis of bean yellow dwarf virus, a geminivirus of the genus Mastrevirus that is adapted to dicotyledonous plants. J. Gen. Virol. 79, 2265-2274. doi:10.1099/0022-1317-79-9-2265.
Lozano-Duran, R., Rosas-Diaz, T., Luna, A. P., and Bejarano, E. R. (2011). Identification of host genes involved in geminivirus infection using a reverse genetics approach. PLoS One 6, e22383. doi: 10.1371/j ournal.pone.0022383.
Luftig, M. A. (2014). Viruses and the DNA Damage Response: Activation and Antagonism. Annu. Rev. Virol. 1, 605-625. doi:10.1146/annurev-virology-031413-085548.
Mason, H. S., DeWald, D. B., and Mullet, J. E. (1993). Identification of a methyl jasmonate-responsive domain in the soybean vspB promoter. Plant Cell 5, 241-51. doi:10.1105/tpc.5.3.241.
Mason, H. S., Ball, J. M., Shi, J. J., Jiang, X., Estes, M. K., and Arntzen, C. J. (1996). Expression of Norwalk virus capsid protein in transgenic tobacco and potato and its oral immunogenicity in mice. Proc. Natl. Acad. Sci. 93, 5335-5340. doi:10.1073/pnas.93.11.5335.
Mathew, L. G., Herbst-Kralovetz, M. M., and Mason, H. S. (2014). Norovirus narita 104 virus-like particles expressed in Nicotiana benthamiana induce serum and mucosal immune responses. Biomed Res. Int. 2014, 1-9. doi:10.1155/2014/807539.
Mafić, S., Pegoraro, M., and Noris, E. (2016). The C2 protein of tomato yellow leaf curl Sardinia virus acts as a pathogenicity determinant and a 16-amino acid domain is responsible for inducing a hypersensitive response in plants. Virus Res. 215, 12-19. doi:10.1016/j.virusres.2016.01.014.
Matsuda, R., Abe, T., Fujiuchi, N., Matoba, N., and Fujiwara, K. (2017). Effect of temperature post viral vector inoculation on the amount of hemagglutinin transiently expressed in Nicotiana benthamiana leaves. J. Biosci. Bioeng. 124, 346-350. doi: 10.1016/j.jbiosc.2017.04.007.
Missich, R., Ramirez-Parra, E., and Gutierrez, C. (2000). Relationship of Oligomerization to DNA Binding of Wheat Dwarf Virus RepA and Rep Proteins. Virology 273, 178-188. doi:10.1006/viro.2000.0412.
Moon, K.-B., Lee, J., Kang, S., Kim, M., Mason, H. S., Jeon, J.-H., et al. (2014). Overexpression and self-assembly of virus-like particles in Nicotiana benthamiana by a single-vector DNA replicon system. Appl. Microbiol. Biotechnol. 98, 8281-8290. doi:10.1007/s00253-014-5901-6.
Nandi, S., Kwong, A. T., Holtz, B. R., Erwin, R. L., Marcel, S., and McDonald, K. A. (2016). Techno-economic analysis of a transient plant-based platform for monoclonal antibody production. MAbs 8, 1456-1466. doi:10.1080/19420862.2016.1227901.
Nishimura, M. T., and Dangl, J. L. (2010). Arabidopsis and the plant immune system. Plant J. 61, 1053-1066. doi:10.1111/j.1365-313X.2010.04131.x.
Peyret, H., Gehin, A., Thuenemann, E. C., Blond, D., El Turabi, A., Beales, L., et al. (2015). Tandem fusion of hepatitis B core antigen allows assembly of virus-like particles in bacteria and plants with enhanced capacity to accommodate foreign proteins. PLoS One 10, e0120751. doi:10.1371/journal.pone.0120751.
Phoolcharoen, W., Bhoo, S. H., Lai, H., Ma, J., Arntzen, C. J., Chen, Q., et al. (2011). Expression of an immunogenic Ebola immune complex in Nicotiana benthamiana. Plant Biotechnol. J. 9, 807-16. doi:10.1111/j.1467-7652.2011.00593.x.
Pilartz, M., and Jeske, H. (2003). Mapping of Abutilon Mosaic Geminivirus Minichromosomes. J. Virol. 77, 10808-10818. doi:10.1128/JVI.77.20.10808-10818.2003.
Qian, Y., Hou, H., Shen, Q., Cai, X., Sunter, G., and Zhou, X. (2016). RepA Protein Encoded by Oat dwarf virus Elicits a Temperature-Sensitive Hypersensitive Response-Type Cell Death That Involves Jasmonic Acid-Dependent Signaling. Mol. Plant. Microbe. Interact. 29, 5-21. doi:10.1094/MPMI-07-15-0149-R.
Ruschhaupt, M., Martin, D. P., Lakay, F., Bezuidenhout, M., Rybicki, E. P., Jeske, H., et al. (2013). Replication modes of Maize streak virus mutants lacking RepA or the RepA-pRBR interaction motif. Virology 442, 173-179. doi:10.1016/j.virol.2013.04.012.
Sakamoto, T., Deguchi, M., Brustolini, 0. J., Santos, A. A., Silva, F. F., and Fontes, E. P. (2012). The tomato RLK superfamily: phylogeny and functional predictions about the role of the LRRII-RLK subfamily in antiviral defense. BMC Plant Biol. 12, 229. doi:10.1186/1471-2229-12-229.
Sanders, P. R., Winter, J. A., Barnason, A. R., Rogers, S. G., and Fraley, R. T. (1987). Comparison of cauliflower mosaic virus 35S and nopaline synthase promoters in transgenic plants. Nucleic Acids Res. 15, 1543-58. Available at: http://www.ncbi.nlm.nih.gov/pubmed/3029718 [Accessed Oct. 17, 2018].
Santi, L., Batchelor, L., Huang, Z., Hjelm, B., Kilbourne, J., Arntzen, C. J., et al. (2008). An efficient plant viral expression system generating orally immunogenic Norwalk virus-like particles. Vaccine 26, 1846-54. doi:10.1016/j.vaccine.2008.01.053.
Segonzac, C., and Zipfel, C. (2011). Activation of plant pattern-recognition receptors by bacteria. Curr. Opin. Microbiol. 14, 54-61. doi:10.1016/j.mib.2010.12.005.
Strasser, R., Altmann, F., and Steinkellner, H. (2014). Controlled glycosylation of plant-produced recombinant proteins. Curr. Opin. Biotechnol. 30, 95-100. doi: 10.1016/j.copbio.2014.06.008.
Sugio, T., Matsuura, H., Matsui, T., Matsunaga, M., Nosho, T., Kanaya, S., et al. (2010). Effect of the sequence context of the AUG initiation codon on the rate of translation in dicotyledonous and monocotyledonous plant cells. J. Biosci. Bioeng. 109, 170-173. doi:10.1016/j jbiosc.2009.07.009.
Takeuchi, O., and Akira, S. (2009). Innate immunity to virus infection. Immunol. Rev. 227, 75-86. doi:10.1111/j.1600-065X.2008.00737.x.
Thornburg, R. W., An, G., Cleveland, T. E., Johnson, R., and Ryan, C. A. (1987). Wound-inducible expression of a potato inhibitor II-chloramphenicol acetyltransferase gene fusion in transgenic tobacco plants. Proc. Natl. Acad. Sci. 84, 744-748. doi:10.1073/pnas.84.3.744.
Tusé, D., Tu, T., and McDonald, K. A. (2014). Manufacturing Economics of Plant-Made Biologics: Case Studies in Therapeutic and Industrial Enzymes. Biomed Res. Int. 2014, 1-16. doi:10.1155/2014/256135.
van Wezel, R., Dong, X., Blake, P., Stanley, J., and Hong, Y. (2002). Differential roles of geminivirus Rep and AC4 (C4) in the induction of necrosis in Nicotiana benthamiana. Mol. Plant Pathol. 3, 461-71. doi:10.1046/j.1364-3703.2002.00141.x.
Wroblewski, T., Tomczak, A., and Michelmore, R. (2005). Optimization of Agrobacterium-mediated transient assays of gene expression in lettuce, tomato and Arabidopsis. Plant Biotechnol. J. 3, 259-273. doi:10.1111/j.1467-7652.2005.00123.x.
Zeitlin, L., Pettitt, J., Scully, C., Bohorova, N., Kim, D., Pauly, M., et al. (2011). Enhanced potency of a fucose-free monoclonal antibody being developed as an Ebola virus immunoprotectant. Proc. Natl. Acad. Sci. 108, 20690-20694. doi:10.1073/pnas.1108360108.
Zhou, J., Yu, J.-Q., and Chen, Z. (2014). The perplexing role of autophagy in plant innate immune responses. Mol. Plant Pathol. 15, 637-645. doi:10.1111/mpp.12118.
Zhou, Y.-C. Y.-C., Garrido-Ramirez, E. R., Sudarshana, M. R., Yendluri, S., and Gilbertson, R. L. (2007). The N-terminus of the Begomovirus nuclear shuttle protein (BV1) determines virulence or avirulence in Phaseolus vulgaris. Mol. Plant. Microbe. Interact. 20, 1523-1534. doi:10.1094/MPMI-20-12-1523.
Zvereva, A. S., and Pooggin, M. M. (2012). Silencing and innate immunity in plant defense against viral and non-viral pathogens. Viruses 4, 2578-2597. doi:10.3390/v4112578.

1. A T-DNA binary vector having a T-DNA region comprising a replicon cassette and an expression cassette, wherein the replicon cassette comprises a Rep/RepA gene with a mutation in the translation initiation site at position −3 and the nucleic acid at position −3 is not A or G.

2. The T-DNA region of claim 1, wherein the translation initiation site sequence of the mutated Rep/RepA gene is CACATG.

3. A T-DNA region of a T-DNA binary vector comprising:
a sequence encoding RepA and/or a sequence encoding Rep; and
a sequence encoding the promoter of ubiquitin-3 from potato with ubiquitin fusion.

4. (canceled)

5. A replicating geminiviral expression system comprising:
a first cloning vector with a T-DNA region comprising:
a sequence encoding Rep; and
a sequence encoding the promoter of ubiquitin-3 from potato with ubiquitin fusion;
a second cloning vector with a T-DNA region comprising:
a sequence encoding RepA; and
a sequence encoding the promoter of ubiquitin-3 from potato with ubiquitin fusion; and
a third cloning vector with a T-DNA region comprising an expression cassette and no replicon cassette, wherein the expression cassette comprises:
a promoter region;
a 5′ UTR;
a sequence encoding transgene; and
a 3′ UTR.

6. (canceled)

7. The replication geminiviral expression system of claim 5, wherein the promoter region of the third cloning vector comprises the sequence of the cauliflower mosaic virus 35S promoter.

8. The replication geminiviral expression system of claim 5, wherein the 5′ UTR of the third cloning vector comprises a 5′ UTR selected from the group consisting of:
the 5′ UTR of native Nicotiana benthamiana NbPsaK,
the 5′ UTR from barley yellow mosaic virus, and
the 5′ UTR from cowpea mosaic virus.

9. The replication geminiviral expression system of claim 5, wherein the 5′ UTR of the third cloning vector does not comprise the 5′ UTR from tobacco mosaic virus.

10. The replication geminiviral expression system of claim 5, wherein the 5′ UTR of the third cloning vector does not comprise the 5′ UTR from pea enation mosaic virus.

11. The replication geminiviral expression system of claim 5, wherein the 3′ UTR of the third cloning vector does not comprise the 3′ UTR from pea enation mosaic virus.

12. (canceled)

13. The replication geminiviral expression system of claim 5, wherein the 3′ UTR of the third cloning vector comprises the 3′ UTR from barley yellow mosaic virus or the 3′ UTR from cowpea mosaic virus.

14. (canceled)

15. (canceled)

16. The T-DNA binary vector of claim 3 comprising a first expression cassette, a second expression cassette, and a third expression cassette, wherein:
the first expression cassette comprises:
the sequence encoding Rep; and
the sequence encoding the promoter of ubiquitin-3 from potato with ubiquitin fusion;
the second expression cassette comprises:
the sequence encoding RepA; and
the sequence encoding the promoter of ubiquitin-3 from potato with ubiquitin fusion; and
the third expression cassette comprises:
a promoter region;
a 5′ UTR;
a sequence encoding a transgene; and
a 3′ UTR.

17-26. (canceled)

27. A method of expressing a recombinant protein in plant cell, the method comprising:
administering to a plant cell a composition comprising transformed Agrobacterium, wherein the transformed Agrobacterium is transformed with the T-DNA binary vector of claim 1.

28. A method of expressing for a recombinant protein in plant cell, the method comprising:
administering to a plant cell a composition of bacteria transformed with the replicating geminiviral expression system of claim 5, wherein the composition of bacteria comprises:
a first transformed Agrobacterium;
a second transformed Agrobacterium; and
a third transformed Agrobacterium, wherein:
the first transformed Agrobacterium is transformed with the first T-DNA binary vector;
the second transformed Agrobacterium is transformed with the second T-DNA binary vector; and
the third transformed Agrobacterium is transformed with the third T-DNA binary vector, wherein the sequence encoding the transgene is a sequence encoding the recombinant protein.

29. The method of claim 28, wherein the composition of bacteria produces Rep and RepA at a ratio of 1:1.

30. The method of claim 28, wherein the OD₆₀₀ value of the composition of bacteria is less than 0.8.

31. The method of claim 28, wherein the OD₆₀₀ value of the composition of bacteria is 0.4 or less.

32-39. (canceled)

40. A method of expressing a recombinant protein in plant cell, the method comprising administering to a plant cell a composition comprising an Agrobacterium transformed with the T-DNA binary vector of claim 16.

41. The method of claim 40, wherein the composition of bacteria produces Rep and RepA at a ratio of 1:1.

42. The method of claim 27, wherein the OD₆₀₀ value of the composition comprising transformed Agrobacterium is less than 0.8.

43. The method of claim 27, wherein the OD₆₀₀ value of the composition comprising transformed Agrobacterium is 0.4 or less.
